(19) United States
(12) Patent Application Publication
(10) Pub. No.: US 2024/0148728 A1
(43) Pub. Date: May 9, 2024

(54) NOVEL METHOD TO BLOCK INFLAMMATORY CELL DEATH AND IL-1BETA SECRETION CAUSED BY RIBOTOXINS AND UV IRRADIATION USING GENETIC AND CHEMICAL INHIBITORS OF ZAKA AND THE NLRP1 INFLAMMASOME

(71) Applicants: Nanyang Technological University, Singapore (SG); Agency for Science, Technology and Research, Singapore (SG)

(72) Inventors: Lei ZHONG, Singapore (SG); Kim Samirah ROBINSON, Singapore (SG); Gee Ann TOH, Singapore (SG); Zijin SUN, Singapore (SG)

(21) Appl. No.: 18/547,670

(22) PCT Filed: Feb. 23, 2022
(86) PCT No.: PCT/SG2022/050086

§ 371 Date: Aug. 23, 2023

(30) Foreign Application Priority Data
Feb. 23, 2021 (SG) 10202101805W

(51) Int. Cl. A61K 31/506; A61K 31/365; A61K 31/428; A61K 31/519; A61K 31/69; A61K 35/748; A61P 35/00

(52) U.S. Cl. CPC A61K 31/506; A61K 31/365; A61K 31/428; A61K 31/519; A61K 31/69; A61K 35/748; A61P 35/00

(57) ABSTRACT

The present invention is directed to a method of modulating inflammation and/or related complications triggered by ZAKα kinase-activated NLRP1-driven pyroptosis; a compound or composition comprising said compound for use in the method, and use of said compounds in medicament preparation. More particularly, the inflammation is caused by a ribotoxin or UVB irradiation. Inhibition of said ZAKα kinase-activated NLRP1-driven pyroptosis may be used in the prophylaxis or treatment of human airway or skin inflammation and/or related complications, whereas activation of ZAKα kinase-activated NLRP1-driven pyroptosis may be used in the prophylaxis or treatment of cancer.

### CROSS-REFERENCE TO RELATED APPLICATIONS

The present application is a filing under 35 U.S.C. 371 as the National Stage of International Application No. PCT/SG2022/050086, filed Feb. 23, 2022, entitled “NOVEL METHOD TO BLOCK INFLAMMATORY CELL DEATH AND IL-1BETA SECRETION CAUSED BY RIBOTOXINS AND UV IRRADIATION USING GENETIC AND CHEMICAL INHIBITORS OF ZAKA AND THE NLRP1 INFLAMMASOME,” which claims priority to Singapore Application No. 10202101805W filed with the Intellectual Property Office of Singapore on Feb. 23, 2021, both of which are incorporated herein by reference in their entirety for all purposes.

### INCORPORATION BY REFERENCE OF MATERIAL IN ASCII TEXT FILE

This application incorporates by reference the Sequence Listing contained in the following ASCII text file being submitted concurrently herewith:

File name: 4373-19000 SP103684USZBD Sequence Listing ST25; created on Aug. 23, 2023; and having a file size of 32 KB.

The information in the Sequence Listing is incorporated herein in its entirety for all purposes.

### FIELD OF THE INVENTION

The present invention provides a method of modulating inflammation and/or related complications triggered by ZAKα kinase-activated NLRP1-driven pyroptosis; a compound or composition comprising said compound for use in the method, and use of said compounds in medicament preparation. More particularly, the inflammation is caused by a ribotoxin or UVB irradiation. Inhibition of said ZAKα kinase-activated NLRP1-driven pyroptosis may be used in the prophylaxis or treatment of human airway or skin inflammation and/or related complications, whereas activation of ZAKα kinase-activated NLRP1-driven pyroptosis may be used in the prophylaxis or treatment of cancer.

### BACKGROUND OF THE INVENTION

The innate immune system uses germline-encoded sensor proteins to recognize conserved pathogen- or damage-associated molecular patterns (PAMPs and DAMPs) [Morgensen, Clinical Microbiology Reviews 22:240-273 (2009); Takeuchi, O. and Akira, S., Cell 140:805-820 (2010)]. However, many of these molecules are also present in commensal microbial species or in normal host tissues, making the distinction between pathogenic and non-pathogenic molecules challenging. As a result, multicellular organisms also detect pathogen-induced disruptions of essential cellular processes, rather than the mere presence of foreign molecules [Lopes Fischer et al., Nat Microbiol 5:14-26 (2020); Stuart et al., Nat Rev Immunol 13:199-206 (2013)). Metazoan NACHT, LRR, and PYD domain-containing proteins (NLRPs) assemble the inflammasome complex in response to infection and injuries, leading to an inflammatory form of cell death known as pyroptosis characterized by caspase-1 activation, IL-1 secretion and GSDMD pore formation (respond to pathogens and damage, particularly those that have gained access to the cytosol [Broz, P. and Dixit V. M., Nat. Rev. Immunol 16:407-420(2016); Rathinam, V. A. K. and Fitzgerald, K. A., Cell 165:792-800 (2016); Vanaja, S. K. et al., Trends Cell Biol. 25:208-31 (2015); van de Veerdonk, F. L., et al., Trends Immunol 32:110-116 (2011)]. NLRs can directly bind and become activated by a wide array of PAMPs, e.g. bacterial proteins, lipopolysaccharides and viral nucleic acids [Bauernfeind and Hornung, EMBO Mol Med 5:814-826 (2013); Storek and Monack, Immunol Rev 265:112-129 (2015); Zhao and Shao, Curr Opin Microbiol 29:37-42 (2016)]. However, the ability to function as effector-triggered pathogen sensors, i.e. to sense the telltale effects of pathogen attack on essential cellular processes, has only been demonstrated for a small number of metazoan NLRs [Chen and Chen, Nature 564:71-76 (2018); Gao et al., PNAS U.S.A. 113:E4857-E4866 (2016); Xu et al., Nature 513:237-241 (2014)].

NLRP1 is notable among mammalian NLR sensors due to its unusual domain arrangement and tissue distribution [Mitchell et al., Curr Opin Immunol 60:37-45 (2019); Taabazuing et al., Immunol Rev 297:13-25 (2020)]. NLRP1 assembles the inflammasome complex through a C-terminal CARD domain and requires two related proteases, DPP8 and DPP9 for auto-inhibition [Okondo et al., Cell Chem Biol 25:262-267.e5 (2018); Zhong et al., J Biol Chem 293:18864-18878 (2018)]. In contrast to other inflammasome sensors such as NLRP3, human NLRP1 is predominantly expressed in the skin and airway epithelia [Robinson et al., Science (2020); Sand et al., Cell Death Dis 9:24 (2018); Zhong et al., Cell 167:187-202.e17 (2016)]. Germline mutations in NLRP1 cause a number of Mendelian diseases characterized by epithelial hyperplasia and dyskeratosis, with only the most severe cases demonstrating periodic fever and systemic auto-inflammation seen in other inflammasome disorders [Drutman et al., PNAS U.S.A 116:19055-19063 (2019); Grandemange et al., Ann Rheum Dis 76:1191-1198 (2017); Zhong et al., Cell 167:178-202.e17 (2016)]. Thus, human NLRP1 plays a unique role in skin immunity that is not shared by other inflammasome sensors or its rodent homologs [Sand, J. et al., Cell Death Dis 9:24 (2018); Joost, S. et al., Cell Syst 3:221-237.e9 (2016)]. Recently our groups and others have described the first bona fide pathogen triggers for human NLRP1, ie. enteroviral 3C proteases and double stranded viral RNA [Bauernfried et al., Science (2020); Robinson et al., Science (2020); Tsu et al., Elife 10 (2021)] and ultraviolet B (UVB) irradiation [Fenini, G. et al., J Invest Dermatol 138:2644-2652 (2018); Sand, J. et al., Cell Death Dis. 9:24 (2018)]. However, it is currently not clear if NLRP1 senses UVB irradiation directly, or responds indirectly to a cellular damage signal induced by UVB. Remarkably none of these triggers activate rodent NLRP1, which lacks the N-terminal extension and has evolved to sense rodent-specific triggers such as anthrax lethal factor and an unknown molecule from Toxoplasma gondii [Cirelli et al., PLoS Pathog 10:e1003927 (2014); Levinsohn et al., PLoS Pathog 8:e1002638 (2012)]. The full repertoire of NLRP1 ligands and the identities of non-viral pathogen(s) sensed by human NLRP1 remain unknown.

There is a need to identify further triggers of NLRP1 in order to devise compositions and methods to modulate inflammatory pathologies caused by NLRP1 induction.

### SUMMARY OF THE INVENTION

The present invention is directed to a method of modulating inflammation and/or related complications triggered by ZAKα kinase-activated NLRP1-driven pyroptosis; a compound or composition comprising said compound for use in the method, and use of said compounds in medicament preparation. More particularly, the inflammation is caused by a ribotoxin or UVB irradiation. Inhibition of said ZAKα kinase-activated NLRP1-driven pyroptosis may be used in the prophylaxis or treatment of human airway or skin inflammation and/or related complications, whereas activation of ZAKα kinase-activated NLRP1-driven pyroptosis may be used in the prophylaxis or treatment of cancer.

According to a first aspect, the present invention provides a composition comprising a ZAKα kinase inhibitor and/or a NLRP1 inhibitor for inhibiting NLRP1-driven pyroptosis in a cell caused by ribosome stalling and/or ribosome collisions within said cell.

In some embodiments of the composition;
a) the ZAKα kinase inhibitor is selected from:
   i) Nilotinib, IUPAC name 4-methyl-N-[3-(4-methylimidazol-1-yl)-5-(trifluoromethyl)phenyl]-3-[(4-pyridin-3-ylpyrimidin-2-yl)amino]benzamide,
   M443 4-methyl-N-[3-(4-methyl-1H-imidazol-1-yl)-5-(trifluoromethyl)phenyl]-3-[[4-[1-(1-oxo-2-propen-1-yl)-3-piperidinyl]-2-pyrimidinyl]amino]-benzamide, or
   5-Z-7-oxozeanol;
   ii) CRISPR-Cas targeting ZAKα or NLRP1, or
   iii) an aptamer; and/or
b) the NLRP1 inhibitor is selected from:
   i) MLN4924, IUPAC name ((1S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate, or hydrochloride salt thereof,
   TAS4464, IUPAC name 7H-Pyrrolo[2,3-d]pyrimidin-4-amine, 7-[5-[(aminosulfonyl)amino]-5-deoxy-beta-D-ribofuranosyl]-5-[2-(2-ethoxy-6-fluorophenyl)ethynyl]-, or hydrochloride salt thereof;
   ZM223, IUPAC name N-[6-[[2-(4-aminophenyl)sulfanylacetyl]amino]-1,3-benzothiazol-2-yl]-4-(trifluoromethyl)benzamide; or
   Bortezomib, IUPAC Name: [(1R)-3-methyl-1-[[(2S)-3-phenyl-2-(pyrazine-2-carbonyl amino)propanoyl]amino]butyl]boronic acid;
   ii) CRISPR Cas, or
   iii) an aptamer.

In some embodiments, the ribosome stalling and/or ribosome collisions are caused by a ZAKα-activating ribotoxin or UVB irradiation.

“CRISPR-Cas” refers to a microbial adaptive immune system that uses RNA-guided nucleases to cleave foreign genetic elements. It comprises clustered regularly interspaced short palindromic repeats (CRISPRs), a CRISPR-associated (Cas) endonuclease and a synthetic guide RNA that can be programmed to identify and introduce a double strand break at a specific site within a targeted gene sequence. The palindromic repeats are interspaced by short variable sequences derived from exogenous DNA targets known as protospacers, and together they constitute the CRISPR RNA (crRNA) array. Within the DNA target, each protospacer is always associated with a protospacer adjacent motif (PAM), which can vary depending on the specific CRISPR system. CRISPR-Cas9 is a specific version of the system referring to use of RNA-guided Cas9 nuclease, originally derived from Streptococcus pyogenes, whereby the target DNA must immediately precede a 5′-NGG PAM. Variations of the CRISPR-Cas9 system are known [Ran F A, et al., Nat. Protoc 8, 2281-2308 (2013); Ran F A, et al., Cell 154, 1380-1389 (2013), incorporated herein by reference] and it is not intended that the present invention be limited to a particular CRISPR-Cas system. CRISPR-Cas could be used to inhibit ZAKα kinase activity or NLRP1 activity generally. The nucleotide sequence of NLRP1 is set forth in SEQ ID NO: 19. The nucleotide sequence of ZAKα kinase is set forth in SEQ ID NO: 20.

Aptamers are molecules that interact with a target nucleic acid or protein, preferably in a specific way. Typically, aptamers are small nucleic acids ranging from 15-50 bases in length that fold into defined secondary and tertiary structures, such as stem-loops or G-quartets. Representative examples of how to make and use aptamers to bind a variety of different target molecules can be found in, for example, U.S. Pat. Nos. 5,476,766 and 6,051,698 (which are hereby incorporated by reference only for this teaching). The secondary structure may inhibit expression of a polypeptide encoded by a gene or inhibit the function of a polypeptide itself. Aptamers bind to these specific targets because of electrostatic interactions, hydrophobic interactions, and their complementary shapes. Aptamers of the present disclosure may interact with and block, for example, ZAKα kinase phosphorylation sites on NLRP1; in particular one or both phosphorylation sites comprising PTSTAVL (SEQ ID NO: 16).

In some embodiments, the CRISPR Cas or aptamer targets (a) ZAKα kinase or (b) NLRP1.

In some embodiments, the aptamer targets (a) the kinase domain of ZAKα kinase or (b) one or more ZAKα kinase phosphorylation sites within a sequence motif comprising the amino acid sequence PTSTAVL (SEQ ID NO: 16) of NLRP1. There are two sites in NLRP1. Amino acids 111-117 (PTSTAVL) and amino acids 177-183 (PTSTAVL). The second site has greater functional significance.

The amino acid sequence of ZAKα kinase is set forth in SEQ ID NO: 17.

In some embodiments, the sequence motif comprises amino acids T178, S179 and T180 of the amino acid sequence of NLRP1 set forth in SEQ ID NO: 18.

According to a second aspect, the present invention provides a composition to activate NLRP1-driven pyroptosis in a cell, comprising a compound that causes ribosome stalling and/or ribosome collisions in said cell.

In some embodiments, the compound activates ZAKα kinase.

In some embodiments, the compound is a ribotoxin or a ribotoxin conjugated to a targeting molecule such as an antibody.

In some embodiments, the compound is a ribotoxin selected from the group comprising Anisomycin, Hygromycin, Deoxynivalenol, Diphtheria Toxin and Exotoxin A (from Pseudomonas aeruginosa).

According to a third aspect, the present invention provides use of a composition according to the first aspect in the manufacture of a medicament for the treatment of an inflammatory pathology triggered by NLRP1-driven pyroptosis caused by ribosome stalling and/or ribosome collisions.

In some embodiments, the ribosome stalling and/or ribosome collisions are caused by a ZAKα-activating ribotoxin or UVB irradiation.

In some embodiments, the inflammatory pathology is due to a microbial ribotoxin.

In some embodiments, the inflammatory pathology is
i) sunburn caused by UVB irradiation, or
ii) UV-driven skin photosensitivity.

In some embodiments, the skin photosensitivity is in a subject with lupus erythematosus or bullous pemphigoid and serious solar urticaria

According to a fourth aspect, the present invention provides use of a composition according to the second aspect in the manufacture of a medicament for activating NLRP1-driven pyroptosis.
In some embodiments, the medicament for activating NLRP1-driven pyroptosis is for the treatment of cancer.

According to a fifth aspect, the present invention provides a method of treating an inflammatory pathology triggered by ribosome stalling and/or ribosome collisions, the method comprising administering to a subject in need thereof an efficacious amount of a composition of the first aspect.

In some embodiments, the ribosome stalling and/or ribosome collisions are caused by a ZAKα-activating ribotoxin.

In some embodiments, the ribotoxin is produced by Corynebacterium Diphtheria or Pseudomonas aeruginosa infection, or is a fungal deoxynivalenol toxin.

According to a sixth aspect, the present invention provides a method of treating a sunburn or skin photosensitivity disorder caused by UVB irradiation, the method comprising administering to a subject in need thereof an efficacious amount of a composition of the first aspect.

In some embodiments, the UVB irradiation is from a solar or an artificial source.

In some embodiments a CRISPR-Cas targeting (a) ZAKα kinase or (b) NLRP1 may be delivered transdermally by, for example, a microneedle patch. A person skilled in the art would know of methods for transdermal delivery of bioactive agents.

### BRIEF DESCRIPTION OF THE FIGURES

All significance values were calculated based on ANOVA from three biological replicates, with each treatment/transfection considered a single replicate. Significance values were indicated as: n.s (non-significant), **P<0.01, ***P<0.001, ****P<0.0001.

FIG. 1A-G show chemical inhibitors that stall elongating ribosomes cause NLRP1-driven pyroptosis in human cells. A, Percentage of 293T-ASC-GFP-NLRP1 cells with ASC-GFP specks after treatment with a small chemical screen: Talabostat (VbP, 5 μM), Anisomycin (ANS, 1 μM), Harringtonine (HTN, 1 μM), lactimidomycin (LTM, 1 μg/ml), Camptothecin (CPT, 1 μM), Etoposide (EPEG, 1 μM), Staurosporine (STS, 1 μM), Hydrogen peroxide (H2O2, 100 μM), Thapsigargin (TGN, 2 μM), lonomycin (IONO, 1 μM), G10 (5 μM), 5Z-7-Oxozeaenol (5z7, 5 μM), Leu-Leu methyl ester, (LLOMe, 5 μM), Bortezomib (BTZ, 1 μM), Pevonedistat (MLN4924, 0.25 μM). Data are the mean+/−s.e.m of 3 independent samples, significance values areas indicated ****p<0.0001 compared to untreated sample (two-way ANOVA). B, Schematic showing where ANS and LTM inhibit the eukaryotic ribosome. C, Representative brightfield images of N-TERT keratinocytes showing classic pyroptotic (yellow arrows) and apoptotic (black arrows) morphology after VbP (2 μM), ANS (1 μM), Puromycin (PURO, 2 μM). Scale bar represents 100 μM. D, Brightfield and fluorescence images of N-TERT-ASC-GFP cells stained for propidium iodide (PI), and Annexin V and treated with VbP (2 μM), ANS (1 μM), PURO (2 μM). Scale bar represents 50 μM. E, Stacked bar graphs showing percentage of live, apoptotic and pyroptotic cells after VbP (2 μM), ANS (1 μM), PURO, 2 μM. Data are the mean+/−s.e.m of 3 independent samples. F, Immunoblot analysis of apoptotic (cleaved PARP1, cleaved CASP3) and pyroptotic (GSDMD-FL, GSDMD-NT, pro-IL1β, IL1β p17 and ASC) markers following chemical treatment. G, Secreted IL1β cytokine levels following treatment of N-TERT cells with VbP (2 μM), ANS (1 μM), HYGRO (280 μM), CHX (10 μM), HTN (1 μM), Blasticidin (BLA, 5 μg/mL), G418 (150 μM), Puromycin (PURO, 2 μM), Tigecycline (TGCL, 10 μM). Data are the mean+/−s.e.m of 3 independent samples, significance values areas indicated ****p<0.0001 compared to untreated sample (two-way ANOVA).

FIG. 2A-H show that chemical inhibitors that stall elongating ribosomes cause NLRP1-driven pyroptosis in human cells. A, Representative images for FIG. 1A B, Percentage of 293T-ASC-GFP-NLRP1 cells with ASC-GFP specks after increasing levels of ANS (1 μM). Data are the mean+/−s.e.m of 3 independent samples, **P<0.01, ****P<0.0001 (two-way ANOVA) compared to untreated control. C, Brightfield and fluorescence images of N-TERT-ASC-GFP cells stained for propidium iodide (PI), and Annexin V and treated with LTN (1 μM), HYGRO (150 μM), HTN (1 μM). Scale bar represents 50 μM. D, Stacked bar graphs showing percentage of live, apoptotic and pyroptotic cells after treatment of N-TERT-ASC-GFP cells with LTN (1 μM), HYGRO (150 μM), HTN (1 μM). E, Immunoblot analysis of DSS crosslinked ASC, ASC and pro-IL1β in the lysate or secreted IL1β in the media. F, Representative images for Deoxynivalenol (DON, 10 μM) treatment in NLRP1 KO+ NLRP1 WT cells. G, Secreted IL1β cytokine levels following treatment of N-TERT cells with indicated ANS doses. Data are the mean+/−s.e.m of 3 independent samples, ****P<0.0001 (two-way ANOVA) as compared to untreated control. H, Secreted IL1β cytokine levels following treatment of N-TERT cells with indicated ANS and Emetine doses. Data are the mean+/−s.e.m of 3 independent samples, n.s.=non-significant, ****P<0.0001 (two-way ANOVA).

FIG. 3A-G show that diphtheria Toxin and Pseudomonas aeruginosa Exotoxin A cause pyroptosis and IL1β in primary human cells. A, Schematic showing that Diphtheria Toxin (DT) and Pseudomonas aeruginosa Exotoxin A (ExoTA) inactivate elongation factor 2 (EEF2) preventing transfer of nascent peptide from A-site to P-site. B, Brightfield and fluorescence images of N-TERT-ASC-GFP cells stained for PI, and Annexin V and treated with TNFα+DT (0.01 μg/ml) or TNFα+ExoTA (5 μg/ml). Scale bar represents 50 μM. C, Stacked bar graphs showing percentage of live, apoptotic and pyroptotic cells after treated with TNFα+DT (0.01 μg/ml) or TNFα+ExoTA (5 μg/ml). D, Immunoblot analysis of apoptotic (cleaved PARP1, cleaved CASP3) and pyroptotic (pro-IL1β, IL1β p17) markers following treatment with TNFα+DT (0.01 μg/ml) or TNFα+ExoTA (5 μg/ml). E, Cleaved GSDMD-NT immunostaining of 3D organotypic skin treated with VbP (2 μM) or TNFα+ExoTA (0.01 μg/ml). Scale bar represents 100 μM. Inset shows a higher magnification of membrane enriched GSDMD-NT signal (black arrows). F, Principal analysis and hierarchical clustering of profiled secreted chemokine/cytokine profiles induced in 3D organotypic skin by VbP (2 μM), ANS (1 μM) and TNFα+DT (0.01 μM). G, Secreted IL1β and IL18 cytokine levels from 3D organotypic skin as measured by ELISA. Data are the mean+/−s.e.m of 3 independent samples, significance values areas indicated ****p<0.0001, **p<0.005 compared to the indicated untreated sample (two-way ANOVA).

FIG. 4A-E show that pyroptosis in primary human keratinocytes caused by inhibition of protein synthesis. A, Representative images following treatment of primary keratinocytes with VbB (2 μM), ANS (1 μM), HYGRO (150 μM), TNFα+DT (0.01 μM), PURO (2 μM). Yellow arrows indicate pyroptosis and black arrows indicate apoptosis. Scale bar represents 50 μM. B, Immunoblot analysis of cleaved caspase 3, DSS crosslinked ASC, pro-IL1β in the lysate or secreted IL113 in the media following VbP (2 μM), ANS (1 μM) treatment in the N-TERTS. C, Immunoblot analysis of DSS crosslinked ASC, following TNFα+DT (0.01 μg/ml) or TNFα+ExoTA (5 μg/ml) in the N-TERTS. E, H&E staining and cleaved GSDMD-NT immunostaining of 3D organotypic skin treated with VbP (2 μM), ANS (1 μM), TNFα+DT (0.01 μg/ml) or TNFα+ExoTA (5 μg/ml). Scale bar represents 100 μM. E, Heat map of profiled secreted chemokine/cytokines induced in 3D organotypic skin by VbP (2 μM), ANS (1 μM), TNFα+DT (0.01 μg/ml) and PURO (2 μM).

FIG. 5A-C show that pyroptosis in NHBE and HAEC cells caused by inhibition of protein synthesis. A, Immunoblot analysis of pro-IL1β in the lysate or secreted IL1β in the media following VbP (2 μM), ANS (1 μM) treatment in primary NHBE cells. b, Immunoblot analysis of cleaved GSDMD-NT and GSDMD-FL in primary HAEC. c, LDH activity assay of HAEC following treatment with VbP (2 μM) and ANS (1 μM). Data are the mean+/−s.e.m of 3 independent samples, *P<0.05, **P<0.01 (two-way ANOVA) compared to untreated control.

FIG. 6A-G show that human NLRP1, but not NLRP3 or CARDS, drives pyroptosis in response to ribosome stalling/collisions. A, Representative brightfield images of NLRP1 WT and NLRP1 KO N-TERT either untreated of after treatment with VbP (2 μM), ANS (1 μM), TNFα+DT (0.01 μg/ml) or TNFα+ExoTA (5 μg/ml). Yellow arrows indicate pyroptosis, black arrows indicate apoptosis. Scale bar represents 50 μM. B, Stacked bar graphs showing percentage of live, apoptotic and pyroptotic cells in WT NLRP1 and NLRP1 KO N-TERT either untreated of after treatment with VbP (2 μM), ANS (1 μM), TNFα+DT (0.01 μg/ml) or TNFα+ExoTA (5 μg/ml). C, Immunoblot analysis of apoptotic (cleaved CASP3) and pyroptotic (pro-IL1β, IL1β p17) markers following ANS (1 μM), TNFα+DT (0.01 μg/ml) or untreated NLRP1, NLRP3, ASC, CASP1 and GSDMD KO N-TERT. D, Heat map of secreted IL1β cytokine levels from WT, NLRP1 KO, NLRP3 KO, ASC KO, CASP1 KO, GSDMD KO N-TERT treated with VbP (2 μM), ANS (1 μM), HYGRO (150 μg/ml), CHX (10 μM), LTM (5 μM), TNFα+DT (0.01 μM) or TNFα+ExoTA (5 μg/ml) as measured by ELISA. Black is equal to high expression and grey is equal to low expression of IL1β. E, Immunoblot analysis of apoptotic (cleaved CASP3) and pyroptotic (pro-IL1β, IL1β p17) markers following ANS (1 μM), TNFα+ExoTA (0.01 μg/ml) or untreated in CASP3+7 double KO or CASP8 KO. F, Representative brightfield images of NLRP1 KO and NLRP1 KO+NLRP1 WT N-TERT either untreated or after treatment with VbP (2 μM), ANS (1 μM). Scale bar represents 50 μM. Yellow arrows indicate pyroptosis. G, Stacked bar graphs showing percentage of live, apoptotic and pyroptotic cells in NLRP1 WT and NLRP1 KO and NLRP1 KO+NLRP1 WT N-TERT either untreated of after treatment with VbP (2 μM), ANS (1 μM) or TNFα+DT (0.01 μM).

FIG. 7A-F show NTERT KO validation. A, Representative brightfield images of NLRP1 WT, NLRP1 KO, NLRP3 KO, ASC KO, CASP1 KO, GSDMD KO N-TERT cell lines treated with VbP (2 μM), ANS (1 μM), HYGRO (150 μg/ml), CHX (10 μM), LTM (5 μM), TNFα+ExoTA (5 μg/ml) or TNFα+DT (0.01 μg/ml). Yellow arrows point to pyroptotic cells. Scale bar represents 50 μM. B, Immunoblot analysis of cleaved GSDMD-NT, ASC DSS crosslinking and IL1β in the media in N-TERT either untreated or treated with HYGRO. C, Immunoblot to validate CASP3 levels in CASP3 KO1/KO2/KO3, black indicates which CASP3 KOs were used. D, Immunoblot to validate GSDMD, CASP1 and ASC levels in ASC, CASP1 and GSDMD KO cell lines. E, Immunoblot to validate CASP8 levels in CASP8 KO2. F, KO scores of NLRP1, NLRP3 and CASP7 KO lines from genomic DNA sequencing.

FIG. 8A-G show KO validations. A, Brightfield and PI stained MV4-11 cells following treatment with VbP (2 μM), ANS (1 μM), TNFα+DT (0.01 μg/ml). Scale bar represents 200 μM. B, Immunoblot analysis of cleaved GSDMD-NT and GSDMD-FL, cleaved CASP3, CARD8-FL, CARD8-NT and CARD8-CT in MV-4-11 cells treated with VbP (2 μM), ANS (1 μM), TNFα+DT (0.01 μg/ml). C, Immunoblot analysis of FL-GSDMD, cleaved GSDMD-NT, FL-CARD8 and CT-CARD8 in ASC, NLRP1, CARD8, CASP1 KOs, NLRP1+CARD8 DKO and WT control with ANS (1 μM) or WT control without ANS in primary HAEC. D, LDH activity assay of WT, NLRP1 KO CARD8 KO and NLRP1+CARD8 DKO HAEC following treatment with VbP, ANS or left untreated. Data are the mean+/−s.e.m of 3 independent samples, n.s.=non-significant, (two-way ANOVA) compared to untreated control E, Representative brightfield images of NLRP1 WT, NLRP1 KO, NLRP1 KO+NLRP1 cell lines treated with VbP (2 μM), ANS (1 μM), HYGRO (150 μM), CHX (10 μM), LTM (1 μM) or TNFα+DT (0.01 μM). Yellow arrows point to pyroptotic cells. Scale bar represents 50 μM. F, Immunoblot analysis of DSS crosslinked ASC, and secreted p17 IL1β in NLRP1 WT, NLRP1 KO and NLRP1 KO+NLRP1 cells treated with VbP (2 μM) and ANS (1 μM). G, Immunoblot analysis of cleaved CASP3, pro-IL1β and secreted p17 IL1β in NLRP1 WT, NLRP1 KO and NLRP1 KO+NLRP1 cells treated with VbP (1 μM) or TNFα+DT (0.01 μM).

FIG. 9A-F show that human NLRP1 responds to ribosome stalling/collisions via a short species-specific disordered loop. A, Schematic showing series of NLRP1 mutants generated to lack either PYD or the individual disordered loops (DR1-3) B, Percentage of ASC-GFP specks after treatment with VbP (2 μM) or ANS (1 μM) in 293T-ASC-GFP cells expressing either full length NLRP1 (a.a. 1-1474), or NLRP1 lacking the PYD (a.a. 86-1474), PYD+DR1 (a.a. 130-1474) region or PYD+DR1-3 (a.a. 254-1474). Data are the mean+/−s.e.m of 3 independent samples, n.s.=non-significant, ****P<0.0001 (two-way ANOVA). C, Representative brightfield images of NLRP1 KO+NLRP1 WT or NLRP1 KO+NLRP1Δ(PYD+DR1) N-TERT either untreated or after treatment with VbP (2 μM), ANS (1 μM). D, IL1β ELISA after treatment with VbP (2 μM) or ANS (1 μM) or TNFα+ExoTA (0.01 μg/ml) in NTERT cells expressing either full length NLRP1 (a.a. 1-1474), or NLRP1 lacking the PYD (a.a. 86-1474), PYD+DR1 (a.a. 130-1474) region or lacking DR1 region only (a.a 1-86+a.a. 130-1474). Data are the mean+/−s.e.m of 3 independent samples, n.s.=non-significant, ****P<0.0001 (two-way ANOVA). E, Immunoblot analysis of NLRP1-NT, FLAG (NLRP1-CT) pro-IL1β or IL1β p17 in the media following ANS (1 μM) or TNFα+ExoTA (0.01 μg/ml) in NLRP1 KO N-TERT rescued with either full length NLRP1 (a.a. 1-1474) or NLRP1 lacking PYD+DR1 (a.a. 130-1474). F, Secreted IL1β cytokine levels following treatment of N-TERT cells with DMSO, ANS (1 μM), TNFα+DT (0.01 μM) in combination with BTZ (0.5 μM) or MLN4924 (0.25 μM) or left untreated. Data are the mean+/−s.e.m of 3 independent samples, n.s.=non-significant, ****P<0.0001 (two-way ANOVA).

FIG. 10A-G show the mechanism of ribosome stalling-dependent NLRP1 activation. A, DPP8/9 enzyme assay in 293T-ASC-GFP-NLRP1 cell line with reconstituted DPP9 and either DMSO, VbP (2 μM), ANS (1 μM) or HTN (1 μM) treatment. Data are the mean+/−s.e.m of 3 independent samples, n.s.=non-significant, (two-way ANOVA). B, Immunoblot following SDS-PAGE or Native-PAGE of 293T-NLRP1-FLAG lysates treated with the indicated drugs. Cells were harvested 5 hours post drug treatment. C, Immunoblot of transduced human NLRP1 (wild-type and variants), CARD8 and murine NLRP1B in NLRP1 KO N/TERT cells. D, IL1β secretion of N/TERT KO cells reconstituted with the NLRP1 or CARD8 in response to anthrax lethal factor toxin (LF). E, IL1β secretion in murine bone marrow derived macrophages of the indicated genotypes treated by VbP, Nigericin or ANS. F, Immunoblot to validate expression of NLRP1 domain deletions expressed in 293T-ASC-GFP. G, Percentage of ASC-GFP specks in 293T-ASC-GFP cells stably expressing full length or NLRP1 (ΔDR1) after treatment with VbP (5 μM) or ANS (2 μM). Data are the mean+/−s.e.m of 3 independent samples, n.s.=non-significant, ****P<0.0001 (two-way ANOVA).

FIG. 11A-G show MLN4924 and BTZ rescue of ribosome stalling dependent NLRP1 activation. A, Representative brightfield images of N-TERT treated with VbP (2 μM), ANS (1 μM), TNFα+DT (0.01 μg/ml), MLN4924 (0.25 μM), BTZ (0.5 μM) or combinations of ANS/TNFα+DT with BTZ/MLN4924 as indicated in the figure. B, Immunoblot analysis of DSS crosslinked ASC, and secreted p17 IL1β in N-TERT cells treated with VbP (1 μM), ANS (1 μM), TNFα+DT (0.01 μg/ml), MLN4924 (0.25 μM), BTZ (0.5 μM) or combinations of ANS/TNFα+DT with BTZ/MLN4924 as indicated in the figure. C, Brightfield and fluorescence images of N-TERT-ASC-GFP cells stained for propidium iodide (PI), and Hoechst and treated TNFα+DT (0.01 μg/ml) or MLN4924 (0.25 μM) and a combination of TNFα+DT and MLN4924. Scale bar represents 50 μM D, Immunoblot analysis of NLRP1-NT, FLAG (NLRP1-FL and NLRP1-CT) and GAPDH markers following ANS (1 μM), VbP (2 μM) and TNFα+DT (0.01 μg/ml) or untreated in 293T-FLAG-NLRP1. E, Percentage of 293T-ASC-GFP-NLRP1 cells with ASC-GFP specks after treatment with DMSO or ANS (1 μM) in combination with BTZ (0.5 μM), MLN4924 (0.25 μM) or left untreated. Data are the mean+/−s.e.m of 3 independent samples, Data are the mean+/−s.e.m of 3 independent samples, n.s.=non-significant, ****P<0.0001 (two-way ANOVA). G, Secreted IL1β cytokine levels following treatment of N-TERT cells with VbP (1 μM) in combination with BTZ (0.5 μM) or MLN4924 (0.25 μM) or left untreated. Data are the mean+/−s.e.m of 3 independent samples, Data are the mean+/−s.e.m of 3 independent samples, n.s.=non-significant, ****P<0.0001 (two-way ANOVA).

FIG. 12A-F show that ribotoxin-induced NLRP1 activation requires ZAKα kinase and a human specific disordered region. A, Ribosomes in NLRP1 KO NTERT+wild-type NLRP1 were pelleted through a sucrose cushion. Pelleted and input lysates were analysed by immunoblot. B, Ribosomes in NLRP1 KO NTERT+wild-type NLRP1 were pelleted through a sucrose gradient. Pelleted and input lysates were analysed by immunoblot. C, Immunoblot analysis of GFP, or ZAKα, with and without phos-tag in NLRP1 KO N-TERT+GFP-NLRP1PYD+DR1 (a.a. 130-1474). D, Schematic showing known ribosome stalling/collision sensing pathways E, Secreted IL113 cytokine levels following treatment of N-TERT cells wild type for ZAKα and ZNF598 or genetic deletion and treated with ANS (1 μM) or VbP (3 μM). Data are the mean+/−s.e.m of 3 independent samples, n.s.=non-significant, ****P<0.0001 (two-way ANOVA). F, Immunoblot analysis of indicated protein in NLRP1 KO N-TERT+GFP-NLRP1PYD+DR1 (a.a. 130-1474). treated with ANS (1 μM) or VbP (3 μM) in combination with nilotinib. Each drug treatment (ANS/VbP) has four samples in the order indicated above the bar graph.

FIG. 13A-G show that N-terminal disordered region regulates NLRP1 activation in response to ribosome stalling. A, Ribosomes in NLRP1 KO NTERT with either NLRP1 WT or NLRP1 Δ(PYD+DR1) were pelleted through a sucrose cushion following treatment with or without ANS. Pelleted and input lysates were analysed by immunoblot. B, Immunoblot to validate ZAKα levels in ZAKα KO1/KO2/KO3/KO4, black indicates which ZAKα KOs were used. C, Schematic showing where ZAKα KO1 (sg1) and KO4 (sg4) guides target the ZAKα and ZAKβ proteins. D, KO scores of ZAKα KO1, ZAKα KO4 and ZNF598 KO3 lines from genomic DNA sequencing. E, Representative brightfield images of ZAKα WT, ZAKα KO1, ZAKα KO4, N-TERT cell lines treated with VbP (2 μM), ANS (1 μM). Yellow arrows point to pyroptotic cells. Scale bar represents 50 μM. F, Representative images of 293T-ASC-GFP-NLRP1 cells with ASC-GFP specks after treatment with ANS (1 μM), VbP (3 μM) or Nilotinib (0.1 μM) or combinations of these 3 drugs. G, Percentage ASC-GFP specks from images represented in F. Data are the mean+/−s.e.m of 3 independent samples, Data are the mean+/−s.e.m of 3 independent samples, n.s.=non-significant, ***P<0.005, ****P<0.0001 (two-way ANOVA).

FIG. 14 shows a schematic diagram of the action of ribotoxins through NLRP1-DR1 phosphorylation.

FIG. 15A-G show A. Immunoblot following SDS-PAGE or PhosTag SDS-PAGE of wild-type or ZAKα KO N-TERT cells expressing NLRP1 DR-GFP. Cells were harvested 2 hours post ANS treatment or UVB irradiation. UVB and ribotoxins cause hyperphosphorylation of the NLRP1 disordered region (DR) in an ZAKalpha dependent manner. pNLRP1-DR is marked on the right. B. GFP immunoblot of NLRP1DR-GFP in NLRP1 KO N-TERT cells treated with the indicated drugs. Cells were harvested 3 hours post treatment. Top panel indicates immunoblot following SDS-PAGE supplemented with PhosTag C. Recombinant SNAP-tagged NLRP1^{DR} was incubated with recombinant ZAKα in a standard kinase reaction for 30 mins. NLRP1 phosphorylation was visualized with SNAP ligand fluorescence (TMR) on a PhosTag-containing SDS-PAGE gel. The recombinant kinase assay shows that ZAKalpha can directly phosphorylate recombinant NLRP1^{DR} (SNAP tagged). Box below shows the phosphorylations site on NLRP1^{DR} mapped by mass spec. The ZAKalpha sites are marked in black. D. IL1β ELISA from NLRP1 KO N-TERT cells expressing full length WT NLRP1 or full length NLRP1 T178A, S179A, T180A (3A) mutant 24 hours after UBV irradiation or ANS treatment. The ZAKalpha site mutant (3A=T178, S179 and T180->AAA) is completely unresponsive to UVB and anisomycin. E. M443 (ZAKalpha inhibitor) is effective at blocking UVB induced NLRP1 activation and IL1beta release in skin explants. * Note that skin explants are more resistant towards UVB induced IL1β secretion, likely due to the thick stratus corneum. Hence a higher dose (mJ/cm²) is required. F. H&E immunostaining of skin explants that were sham, UVB (100 mJ/cm²), UVB (250 mJ/cm²) irradiated either in the presence of M443 or without before harvesting 24 hours later. G. Immunoblot of NLRP1 KO N-TERT cells expressing GFP-tagged NLRP1^{DR} treated with or without ANS alongside various kinase inhibitors. Only M443 and 5-Z-7-oxozeanol can effectively block ANS-triggered NLRP1 hyperphosphorylation. Note that 5-Z-7-oxozeaenol inhibits ZAKα with almost the same potency as its reported target TAK1 and TAKinib, a more selective TAK1 inhibitor does not have an effect on ANS-induced IL1β secretion [Tan et al., Bioorg Med Chem. 25(4):1320-1328 (2017)].

FIG. 16A-C shows that MLN4924 blocks UVB triggered cell death and IL-1beta release. A. Quantification of the percentage of PI positive N-TERT cells during an 18-hour incubation with the indicated drugs. Cells were grown in 0.25 μg/mL PI and imaged at 15 min intervals. B. Quantification of the percentage of PI positive N-TERT cells during an 18-hour incubation with the indicated drugs. Cells were grown at 0.25 μg/mL PI and imaged at 15 min intervals. The most specific NLRP1 agonist known to date, VbP takes at least 5 hours to induce detectable pyroptosis in wild-type N-TERT cells. Hence, the lack of cell death in N-TERT cells within 2 hours should not be interpreted as lack of inflammasome response. The inventors use IL-1β 24 hours post treatment to measure pyroptosis, as this cumulative readout accounts for the different kinetics of pyropsis induced by various triggers. C. IL1β ELISA of N-TERT cell media collected from A and B.

### DETAILED DESCRIPTION OF THE INVENTION

Bibliographic references mentioned in the present specification are for convenience listed at the end of the examples. The whole content of such bibliographic references is herein incorporated by reference for the material contained in them that is discussed in the sentence in which the reference is relied upon.

### Definitions

Certain terms employed in the specification, examples and appended claims are collected here for convenience.

It must be noted that as used herein and in the appended claims, the singular forms “a”, “an”, and “the” include plural reference unless the context clearly dictates otherwise.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

The terms “nucleotide”, “nucleic acid” or “nucleic acid sequence”, as used herein, refer to an oligonucleotide, polynucleotide, or any fragment thereof, to DNA or RNA of genomic or synthetic origin which may be single-stranded or double-stranded and may represent the sense or the antisense strand, to peptide nucleotic acid (PNA), or to any DNA-like or RNA-like material.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

Examples of salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

The term “treatment”, as used in the context of the invention refers to prophylactic, ameliorating, therapeutic or curative treatment.

The term “subject” is herein defined as vertebrate, particularly mammal, more particularly human. For purposes of research, the subject may particularly be at least one animal model, e.g., a mouse, rat and the like. For example, for treatment of airway inflammation and related disorders the subject may be a human with a bacterial or fungal infection that produces a ribotoxin which causes ribosome stalling and/or ribosome collisions. For the treatment of skin inflammation and related disorders the subject may be a human whose skin has been exposed to UVB-irradiation.

A person skilled in the art will appreciate that the present invention may be practiced without undue experimentation according to the methods given herein. The methods, techniques and chemicals are as described in the references given or from protocols in standard biotechnology and molecular biology textbooks. Standard molecular biology techniques known in the art and not specifically described were generally followed as described in Sambrook and Russel, Molecular Cloning: A Laboratory Manual, Cold Springs Harbor Laboratory, New York (2001).

Having now generally described the invention, the same will be more readily understood through reference to the following examples which are provided by way of illustration, and are not intended to be limiting of the present invention.

### EXAMPLES

### Example 1

### Materials and Methods

### Cell Culture and Chemicals

293 Ts (ATCC #CRL-3216), MV-4-11 (ATCC #CRL-9591), mBMDMs (were a kind gift from Linfa Wang, Duke-NUS, Singapore) and normal bronchial epithelial cells (NHBE, Lonza #00-2541) were cultured according to manufacturer's protocols. Immortalised human keratinocytes (N/TERT-1 or N-TERT herein) were provided by H. Rheinwald (MTA) [Dickson et al., Mol Cell Biol 20:1436-1447 (2000); Vyleta et al., PLoS One 7:e36044 (2012)]. Primary human keratinocytes and fibroblasts were derived from the skin of healthy donors and obtained with informed consent from the Asian Skin Biobank. Primary endothelial cells (HAoEC-c or HAEC herein), excised from the ascending and descending aortic arch were purchased from Promocell (#0-12271). All cell lines underwent routine mycoplasma testing with Lonza MycoAlert (Lonza #LT07-118). The following drugs and chemicals were used as part of this study: Staurosporine (STS, MCE, #HY-151141), Puromycin (PURO, Sigma, #P9620), Talabostat (VbP, MCE, #HY-13233), Harringtonine (HTN, MCE, #HY-NO862), Thapsigargin, (TGN, MCE, #HY-13433), Anisomycin (ANS, MCE, #HY1892), Cycloheximide (CHX, Sigma #04859), Lactimidomycin (LTM, Sigma, #506291), Etoposide (EPEG, MCE, #HY13629), Camptothecin (CPT, MCE, #HY16560), ionomycin (IONO, MCE, #HY-13434), G10 (MCE, #HY19711), 5Z-7-Oxozeaenol (5Z7,MCE, #HY12686), Blasticidin (BLA, Sigma, SBR00022), Geneticin (G418, #G8168), Bortezomib (BTZ) and MLN9424 provided by D. Lane, p53 lab, ASTAR, Singapore. Unless indicated, for any treatment with Exotoxin A (SigmaAldrich, 341215) or Diphtheria Toxin (SigmaAldrich, D0564) the cells were primed 24 hours before bacterial toxin treatment with TNFα (Recombinant human TNFα, R&D systems, 210-TA).

### Human Skin Explants

Waste surgical skin tissues from abdomen and breast were collected with appropriate informed consent of the patients and sent to the Asian Skin Biobank (ASB worldwidewebdota-stardotedudotsg/sris/technology-platforms/asian-skin-biobank) at the Skin Research Institute of Singapore (SRIS) (under A*STAR IRB 2020-209). Fresh skin tissue was cleaned in solutions of HBSS with decreasing concentrations of Penicillin, Streptomycin and Fungizone. Then 8-mm skin biopsies were punched and submerged in culture media. Skin explants were immediately irradiated with UVB or treated with VbP.

### 3D Skin Culture

Organotypic cultures were generated by adapting a previously described protocol (Arnette et al., 2016). Briefly, 2 ml of collagen I (4 mg/ml; Corning, #354249) mixed with 7.5×10⁵ human fibroblasts were allowed to polymerize over 1-m1 acellular collagen I in 6-well culture inserts (Falcon, #353102) placed in 6-well deep well plate (Falcon, #355467). After 24 hours, 1×10⁶ primary human keratinocytes were seeded into the inserts and kept submerged in a 3:1 DMEM (Hyclone, #SH30243.01) and F12 (Gibco, #31765035) mixture with 10% FBS (Hyclone, #SV30160.03), 100 U/ml penicillin-streptomycin (Gibco, #15140122), 10 μM Y-27632 (Tocris, #1254), 10 ng/ml EGF (Sigma-Aldrich, #E9644), 1×10⁻¹⁰ M cholera toxin (Enzo, #BML-G117-001), 0.4 μg/ml hydrocortisone (Sigma-Aldrich, #H0888), 0.0243 mg/ml adenine (Sigma-Aldrich, #A2786), 5 μg/ml insulin (Sigma-Aldrich, #12643), 5 μg/ml transferrin (Sigma-Aldrich, #T2036) and 2×10⁻⁹ M 3,3′,5′-triiodo-L-thyronine (Sigma-Aldrich, #T6397). After another 24 hours, the organotypic cultures were then raised at the air-liquid interface and fed with the submerged media (without Y-27632 and EGF) below the insert to induce epidermal differentiation. The air-lifting medium was replaced every 2 days and treatments began 10-14 days after airlifting. Organotypic cultures were then harvested 24 hours after treatment and formalin fixed for 24 hours. Fixed tissues were then embedded into wax for histological purposes.

### UVB and UVA Irradiation

For UVA and UVB irradiation experiments, cells were seeded 24 hours prior to irradiation, and washed once in phosphate buffered saline pH 7.4 before being exposed to indicated dose of irradiation using a BIO-SUN microprocessor controlled, cooled UV irradiation system (BIO-SUN, Vilber). After exposure, PBS was replaced by keratinocyte medium and cells were incubated for indicated time.

### Lambda Phosphatase Dephosphorylation Assay

293T cells were transfected with either the NLRP1PYD fragment (a.a. 1-85) or NLRP1^{DR} fragment (a.a. 86-254) tagged with GFP. After 2 days, cells were treated with ANS for 3 hours, and subsequently harvested and lysed in tris-buffered saline 1% NP-40 with protease inhibitors (Thermo Scientific, #78430). Protein concentration was determined using the Bradford assay (Thermo Scientific, #23200). For each reaction, 40 μg of protein lysate topped up to 40 μl with distilled water was added with 5 μl of 10×NEBuffer for Protein MetalloPhosphatases (PMP) and 5 μl of 10 mM MnCl₂ to make a total reaction volume of 50 μl. 1 μl of Lambda Protein Phosphatase (NEB, P0753S) was added and samples were incubated at 30° C. for 30 minutes. Final reaction products were prepared for SDS-PAGE immunoblotting using the protocol mentioned below.

### Cytokine and Luminex Analysis

To measure secreted cytokine and chemokine levels a human IL1β enzyme linked immunosorbent assay (ELISA) kit (BD, #557953), human IL-18 ELISA kit (MBL, #7620) or an Immune Monitoring 65-Plex Human ProcartaPlex Panel (EPX650-10065-901) were used according to manufacturer's protocols. Further analysis of Luminex or ELISA profiling of samples was performed using heatmap and PCA analysis on Clustervis (biitdotcsdotutdotee/clustvis/) [Somani et al., Proceeding of SSIC (2019)].

### Plasmids and Preparation of Lentiviral Stocks

293T-ASC-GFP, N-TERT-ASC-GFP, N-TERT NLRP1 KO, N-TERT CASP1 KO, N-TERT ASC KO cells were previously described [Zhong et al., J Biol Chem 29318864-18878 (2018)]. All expression plasmids for transient expression were cloned into the pCS2+ vector backbone and cloned using InFusion HD (Clonetech). Constitutive lentiviral expression was performed using pCDH vector constructs (System Biosciences) and packaged using third generation packaging plasmids.

### CRISPR-Cas9 Knockout

Lentiviral Cas9 and guide RNA plasmid (LentiCRISPR-V2, Addgene plasmid #52961) was used to create stable deletions in N-TERT keratinocytes. The sgRNAs target sequences (5′ to 3′) are shown in Table 1.

TABLE 1
List of sgRNA

| | | SEQ |
|---|---|---|
| sgRNA used | | ID |
| (Gene) | Sequence | NO. |
| NLRP1 | GATAGCCCGAGTGCATCGG | 1 |
| ASC | GCTAACGTGCTGCGCGACAT | 2 |
| CASP1 | ACAGACAAGGGTGCTGAACA | 3 |
| NLRP3 | GAATCCCACTGTGATATGCC | 4 |
| GSDMD | AGGTTGACACACTTATAACG | 5 |
| CASP3 | CGTGGTACAGAACTGGACTG | 6 |
| CASP7 | GGTACAAACGAGGACCGGTC | 7 |
| CASP8 | CACAGCATTAGGGACAGGAA | 8 |
| NLRP1 | GGAGTTAAGAGGGTGTCTGG | 9 |
| MAP3K20(ZAKα) sg1 | TGTATGGTTATGGAACCGAG | 10 |
| MAP3K20(ZAKα) sg4 | TGCATGGACCGGAAGACGATG | 11 |
| ZNF598 | CGGCACTCGCGCCGGAACGA | 12 |
| MAPK14(p38α) sg1 | TGATGAAATGACAGGCTACG | 13 |
| MAPK11(p38β) sg1 | CGACGAGCACGTTCAATTCC | 14 |
| MAPK11(p38β) sg2 | ACTCGGCCGGGATCATCCAC | 15 |

Knockout efficiency was tested by immunoblot. Alternatively, Sanger sequencing of genomic DNA and overall editing efficiency determined using the Synthego ICE tool software (Synthego Performance Analysis, ICE Analysis. 2019. v2.0. Synthego).

### Immunoblotting

The following antibodies were used in this study: cleaved PARP1 (Abclonal, #WH162766), Cleaved CASP3 (Abclonal, #WH154646), Full length GSDMD-FL (Abcam, #ab210070), IL1β p17 specific (CST, #83186S), CARD8 (Abcam, CARD8-NT: ab19485, CARD8-CT: ab241186), DPP9 (Abcam, ab226334), c-Myc (Santa Cruz Biotechnology, #sc-40), HA tag (Santa Cruz Biotechnology, #sc-805), GAPDH (Santa Cruz Biotechnology, #sc-47724), ASC (Adipogen, #AL-177), CASP1 (Santa Cruz Biotechnology, #sc-622), 11_1B (R&D systems, #AF-201), FLAG (SigmaAldrich, #F3165), NLRP1 (R&D systems, #AF6788), IL18 (Abcam ab207324), cleaved GSDMD-NT (Asp275) (Cell Signaling Technology, #36425), RPL31 (Abclonal, #WH162766), CASP7 (Abcam, ab255818), CASP8 (Abcam, ab32397), cleaved CASP3 (Cell Signaling Technology, #9664). All horseradish peroxidase (HRP)— conjugated secondary antibodies were purchased from Jackson Immunoresearch (goat anti-mouse IgG: 115-035-166; goat anti-rabbit IgG: 111-035-144; and donkey anti-goat IgG: 705-005-147). Blue-Native PAGE was carried out using the Native-PAGE system (ThermoFisher) with 10-20 μg of total lysate. For SDS-PAGE using whole cell lysates, cells were resuspended in tris-buffered saline 1% NP-40 with protease inhibitors (Thermo Scientific, #78430). Protein concentration was determined using the Bradford assay (Thermo Scientific, #23200) and 20 mg of protein loaded, a part from cleaved GSDMD-NT visualisation where 40 mg of protein was used. All primary antibodies were used at 250 ng/ml. Visualisation of ASC oligomerization was previously described (Robinson et al., 2020). For analysis of IL1β and IL-18 cleavage in the media by immunoblotting, samples were concentrated using filtered centrifugation (Merck, Amicon Ultra, #UFC5003BIK). Protein samples were run using immunoblotting, and then visualized using a ChemiDoc Imaging system (Bio-Rad). PhosTag SDS-PAGE was carried out using homemade 10% SDS-PAGE gel, with addition of Phos-tag Acrylamide (Wako Chemicals, AAL-107) to a final concentration of 30 μM and manganese chloride(II) (Sigma-Aldrich, #63535) to 60 μM.

Cells were directly harvested using Laemmli buffer, lysed with an ultrasonicator, and loaded into the Phos-tag gel to run. Once the run was completed, the polyacrylamide gel was washed in transfer buffer with 10 mM EDTA twice, subsequently washed without EDTA twice, blotted onto 0.45 μm PVDF membranes (Bio-rad), blocked with 3% milk, and incubated with primary and corresponding secondary antibodies.

### Immunohistochemistry

For immunohistochemistry staining of cleaved N-terminal GSDMD a previously established protocol with modifications was used [Wang et al., Cell 180:941-955.e20 (2020)]. Briefly, 5 μm formalin fixed and paraffin embedded sections were deparaffinized and rehydrated through a series of alcohol. The sections were then rehydrated in an antigen retrieval buffer (Dako, Tris-EDTA, pH 9). Sections were then blocked in goat serum for 20 mins at RT before primary antibody incubated overnight at 4° C. Immunostaining was visualised using DAB substrate and chromogen detection kit (Dako, #K3468). Sections were then counterstained with Myer's Hematoxylin.

### Microscopy

N-TERT-ASC-GFP cells were seeded at a cell density of 3000 cells/well of a 96 black well plate (PerkinElmer, CellCarrier-96 Ultra, #6055300). The next morning cells were treated with chemicals for 6 h or treated in the evening for 24 h before staining. 1 hour before observing the cells on the microscope the cells were stained with 1 μg/ml dilution of propidium iodide (PI, Abcam #ab14083), 10 ng/ml dilution of Hoechst 33342 (Life Technologies, #H21492) or stained with Annexin V Alexa Flour 647 (Life technologies, #A23204) according to the manufacturer's protocol. Stained cells were then imaged on a high content screening microscope (Perkin Elmer Operetta CLS imaging system). Images were then stored and analyzed using the Harmony software (Version 6). Fluorescent and brightfield images acquired on the Operetta high content screening microscope were further analyzed using a scoring system to categorize the percentages of live, apoptotic and pyroptotic cells. For 3 fields of view per treatment the number of live cells per field was counted from the merge of the brightfield and ASC-GFP channels. Only cells which contained ASC-GFP throughout the cytoplasm and nucleus were classed as “live”. The number of pyroptotic cells was calculated using the merge of the brightfield and ASC-GFP channels. Only the cells with GFP specks were classed as “pyroptotic”. The number of apoptotic cells was calculated using the merge of the brightfield, ASC-GFP and Annexin V channels. Cells that were stained positive for Annexin V but without a GFP speck were classified as “apoptotic”. Images of ASC-GFP specks were acquired in 3 random fields in 4′,6-diaminidino-2-phenylindole (DAPI, 358 nm/461) and GFP (469 nm/525 nm) channels using the EVOS microscope (FL Auto M5000, #AM F5000) according to the manufacturer's protocol. Quantification method of ASC-GFP specks was previously described in detail [Robinson et al., Science (2020)].

### RNA-Seq

Library preparation, QC and high-throughput sequencing were provided by Macrogen, Singapore. RNA isolation and library preparation was carried out as previously described [Robinson et al., Science (2020)].

### DPP8/9 Activity Assay

293T cells were transfected with vector or wild-type DPP9. DPP9-transfected cells were treated with VbP, Anisomycin (ANS) or Harringtonine (HTN). Cells were lysed in PBS 1% Tween-20, 48 h after transfection and treatments. 0.3 μg of total lysate was then incubated with 0.1 μM uGly-Pro-AMC fluorescence substrate. AMC fluorescence was measured after 30 mins at 25° C. in a 50 μl reaction every minute on a spectrometer and the rate of Gly-Pro-AMC hydrolysis per minute calculated.

### Sucrose Cushions & Sucrose Gradient

Crude cellular ribosome fractions were purified by sedimentation through a 30% sucrose cushion or a 10-30% sucrose gradient. Cells were lysed for 20 min in 15 mM Tris, pH 7.5, 0.5% NP-40, 6 mM MgCl₂, 300 mM NaCl, with 1× protease inhibitors before centrifugation for 10 min at 12000 g, 4° C. The supernatant was then carefully layered onto a 30% sucrose cushion 30% sucrose in 20 mM Tris, pH 7.5, 2 mM MgCl₂, 150 mM KCl and ultra-centrifuged at 34,000 rpm for 24 h using Beckman Coulter Ultracentrifuge, Optima XE. Pellets then washed three times with ice cold PBS and suspended in 100 mM KCl, 5 mM MgCl₂, 20 mM HEPES, pH 7.6, 1 mM DTT and 10 mM NH₄Cl. Purified ribosome fractions were then analysed by immunoblot.

### Statistical Analysis

Statistical analyses were performed using Prism 8 Software (GraphPad). The methods for statistical analysis are included in the figure legends. Error bars show mean values with SEM.

### Example 2

### A Subset of Protein Synthesis Inhibitors Cause NLRP1-Driven Pyroptosis in Human Cells

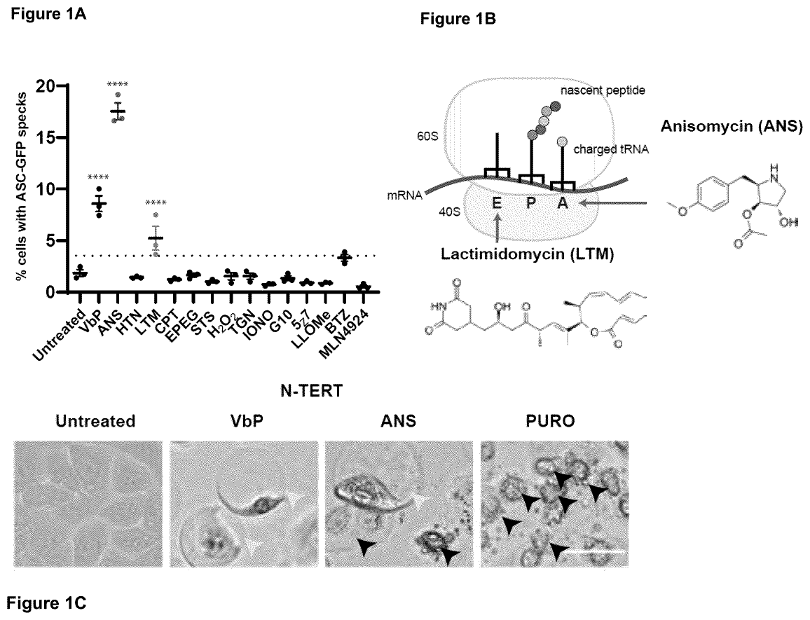

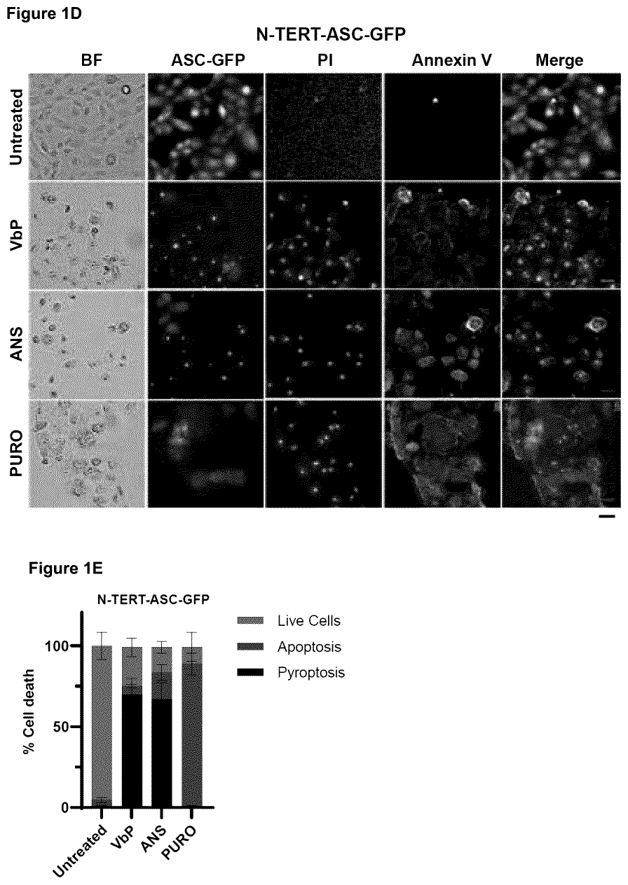

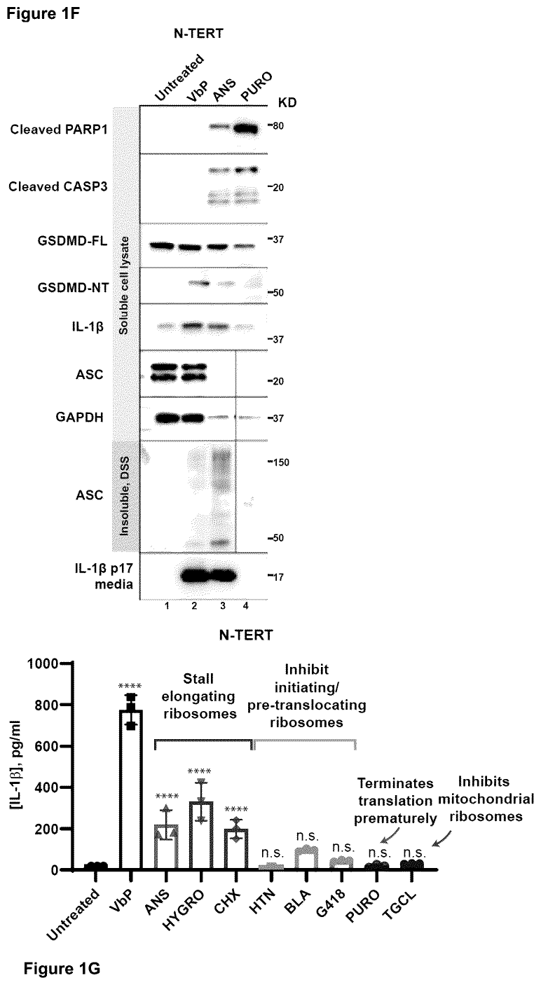

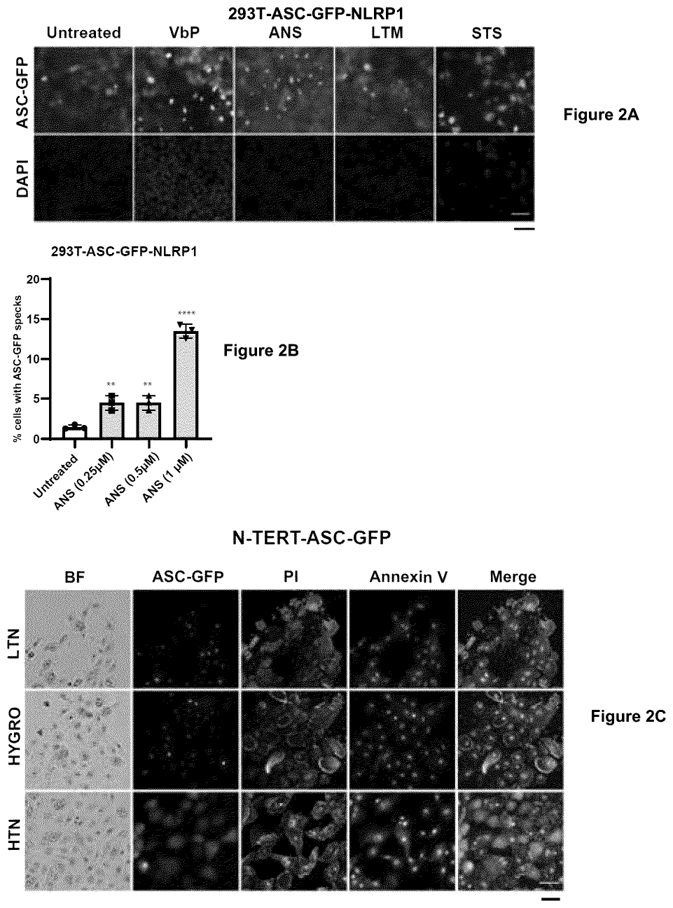

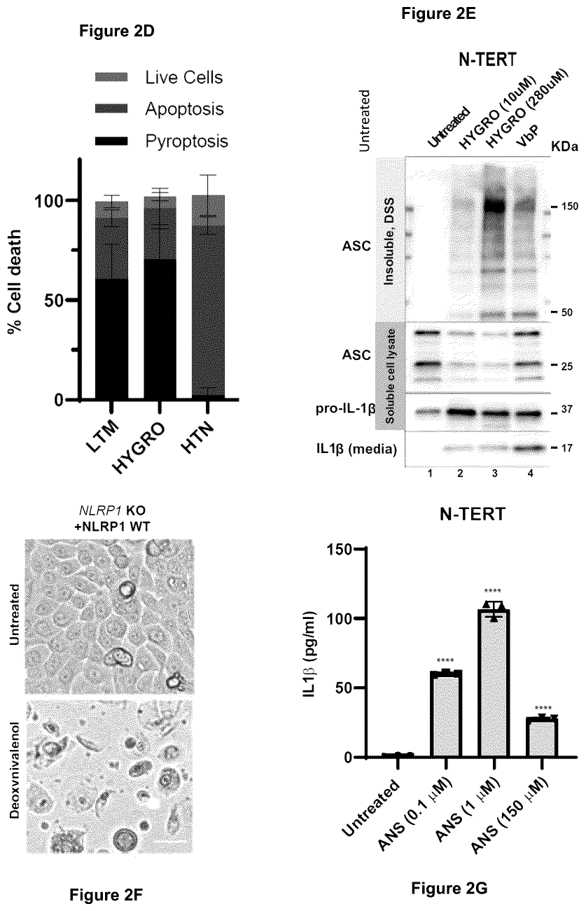

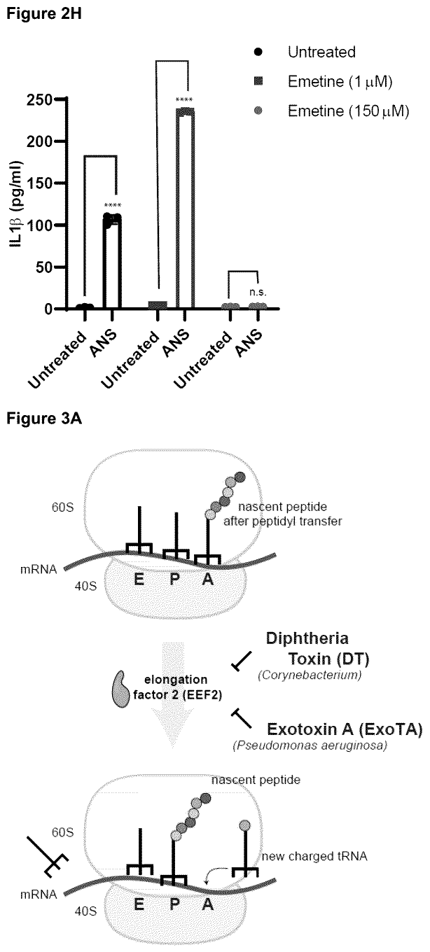

In order to identify additional NLRP1 agonists besides the DPP8/9 inhibitor VbP [Gai et al., Cell Death & Disease 10 (2019); Okondo et al., Nat Chem Biol 13:46-53 (2017); Taabazuing et al., Immunol Rev 297:13-25 (2020); de Vasconcelos et al., Life Sci Alliance 2 (2019); Zhong et al., J Biol Chem 293:18864-18878 (2018)], a number of cytotoxic chemicals were screened for their abilities to induce ASC-GFP speck formation in an inflammasome reporter cell line (293T-ASC-GFP-NLRP1), which stably expressed NLRP1 as the only NLR sensor. This small screen identified two hits, Anisomycin (ANS) and Lactimidomycin (LTM) (FIGS. 1A and 2A). Remarkably, ANS increased the percentage of cells with ASC-GFP specks to a greater extent than VbP at equivalent concentrations (FIG. 1A) and in a dose-dependent manner (FIG. 2B) up to 20 μM. The ability of ANS and LTM to induce ASC-GFP specks was not a by-product of their cytostatic effect, as staurosporine (STS), which caused greater cytotoxicity, failed to induce ASC-GFP specks (FIGS. 1A and 2A).

ANS and LTM are chemically unrelated bacterial secondary metabolites that inhibit the eukaryotic ribosome in the elongation phase (FIG. 1B). ANS binds the peptidyl transfer center of the 60s ribosome subunit and arrests elongating ribosomes at the ‘pre-translocation’ stage [Jiménez and Vázquez, Mechanism of Action of Antieuaryotic and Antiviral Compounds 1-19 (1979)]. On the other hand, LTM occludes the ‘E-site’ of the 60s ribosome and stalls the ribosome right after the first elongation step [Sugawara et al., J Naitbiot 45:1433-1441 (1992)] [Schneider-Poetsch et al., Nat Chem Biol 6:209-217 (2010)]. ANS and LTM are known to induce apoptotic cell death in cultured human cancer cell lines, similar to other translation inhibitors (Schneider-Poetsch et al., Nat Chem Biol 6:209-217 (2010), Macias-Silva et al., Current Chemical Biology 4:124-132 (2010)). ANS has also been shown to activate NLRP3 in murine macrophages [Briard et al., Nature (2020), Vyleta et al., PLoS One 7:336044 (2012)], but the effects of ANS, LTM and related ribosome inhibitors on other inflammasome sensors are not well understood.

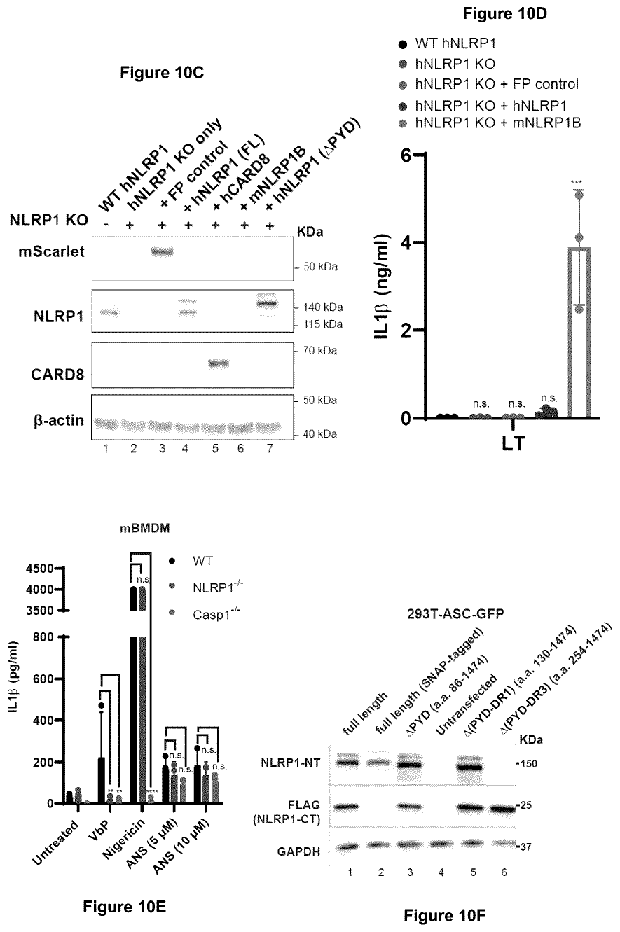

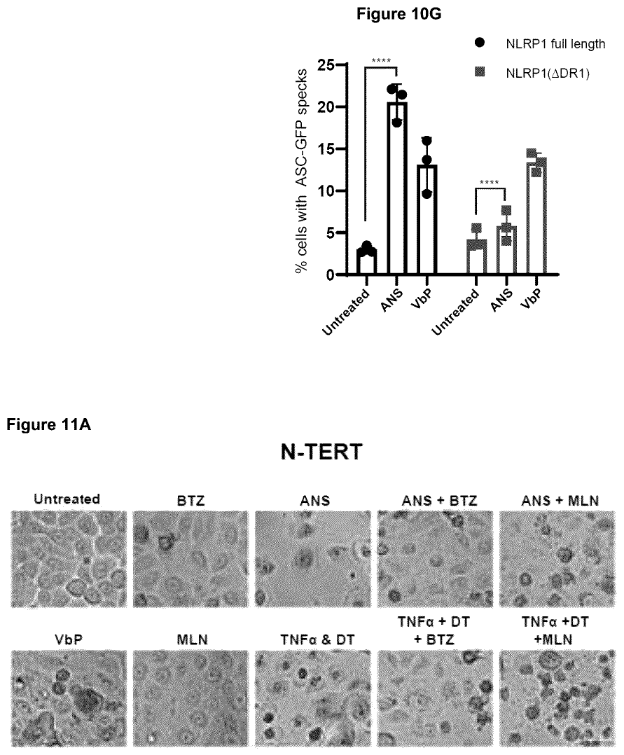

In immortalized keratinocytes (N-TERT), which express NLRP1 endogenously as the most prominent inflammasome sensor (Zhong et al., 2016), both ANS and LTM rapidly induced the morphological hallmarks of inflammasome activation and pyroptotic cell death (white arrows), including 1) ‘ballooning’ of the cell membrane (FIG. 10), 2) aggregation of dispersed ASC-GFP into specks (FIGS. 1D, 2C, and 3) rapid uptake of propidium iodide (PI) (FIGS. 1D and 2C). In addition to ANS and LTM, other inhibitors of elongating ribosomes, such as Hygromycin (HYGRO), another bacterial secondary metabolite and deoxynivalenol (DON), an unrelated fungal toxin, also caused prominent inflammasome activation and pyroptosis (FIGS. 2C, E and F). By examining Annexin V staining and PI inclusion at the single cell level, we found that ANS, LTM and HYGRO caused both apoptosis and pyroptosis in N-TERT cells, with the fraction of pyroptotic cells comparable to that induced by VbP (FIGS. 1 D and E, 2C and D).

ANS- and HYGRO-treated cells displayed cardinal biochemical hallmarks of inflammasome activation, including ASC polymerization, cleavage of GSDMD into the pore-forming p30 fragment (GSDMD-NT) as well as the secretion of mature IL1β p17 into the media (FIGS. 1F and 2E). ANS also activated classical apoptotic cell death as evidenced by cleaved caspase-3 and PARP-1, in agreement with Annexin V and PI staining results (FIGS. 1E and F). Thus, in contrast to VbP, which exclusively triggers pyroptosis, and PURO, which kills cells exclusively via apoptosis, ANS and HYGRO activate both forms of cell death (FIGS. 1F and 2E). Remarkably, none of the inhibitors that block the ribosome at the pre-elongation stages, including harringtonin, blasticidin and G418 caused any pyroptosis in N-TERT cells (FIGS. 1A and G). Thus, in contrast to murine BMDMs [Vyleta et al., PLoS One 7:e36044 (2012)], only inhibitors that can inhibit elongating ribosomes [Vind et al., Mol Cell 78; 700-713.e7 (2020); Wu et al., Cell 182:404-416.e14 (2020), Garreau de Loubresse et al., Nature 513:517-522 (2014)] trigger pyroptosis in NLRP1-expressing human cells.

ANS, HYGRO and DON, by arresting different stages of ribosome movement on mRNA, lead to both stalling and collisions of elongating ribosomes. To distinguish whether stalling or collision serves as the trigger for pyroptosis, N/TERT cells were treated with ultra-high doses of ANS and emetine (150 μM) that are capable of ‘freezing’ most ribosomes and therefore preventing any unaffected trailing ribosomes from colliding with the stalled forerunners. The high concentration of ANS doses led to a significant decrease in IL1β secretion as compared to lower doses (FIG. 2G), and 150 μM emetine completely abrogated ANS-induced IL1β secretion in N-TERTs (FIG. 2H). Thus, although these results cannot completely distinguish the two possibilities, ribosome collision might be a more potent trigger for inflammasome-driven pyroptosis than mere ribosome stalling.

### Example 3

Diphtheria Toxin and Pseudomonas aeruginosa Exotoxin A Cause Pyroptosis and IL-1β Secretion in Primary Human Cells

Two well-known bacterial exotoxins that also inhibit the elongation phase of translation, but do not target the ribosome complex itself were then tested. Diphtheria Toxin (DT), derived from Corynebacterium diphtheriae, the causative agent for diphtheria (Sharma et al., 2019); and exotoxin A (ExoTA) derived from Pseudomonas aeruginosa, an opportunistic human pathogen that causes lung, urinary tract and soft tissue infections [Moradali et al., Frontiers in Cellular and Infection Microbiology 7 (2017)] both inactivate elongation factor 2 (eEF2) via covalent modification. As eEF2 is indispensable for the translocation of peptidyl-tRNA from the A site to the P site in the 60s ribosome, DT and ExoTA effectively shut down translation by inhibiting ribosome translocation (FIG. 2A) [Endo, Ribosome-Inactivating Proeins 151-160 (2014); Robertus and Monzingo, Ribosome-Inactivating Proteins 111-133 (2014)].

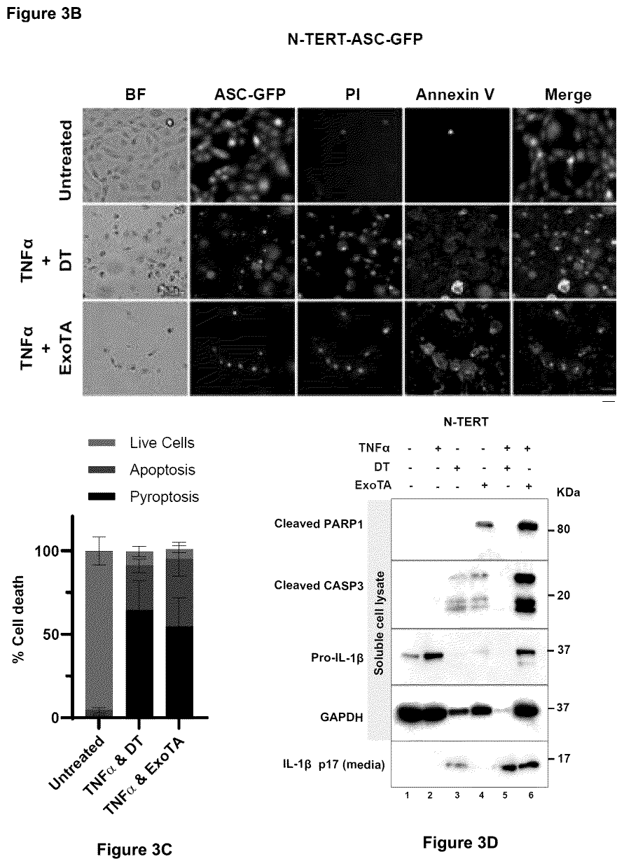

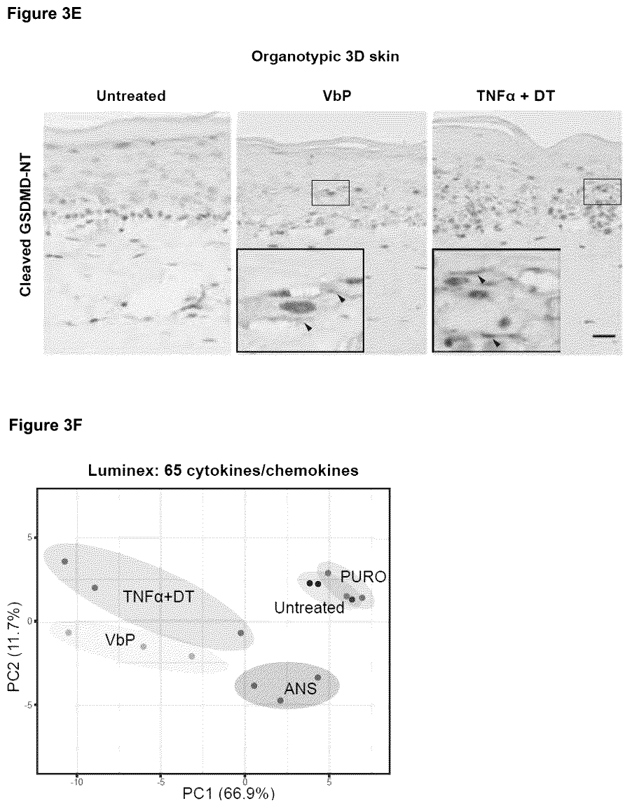

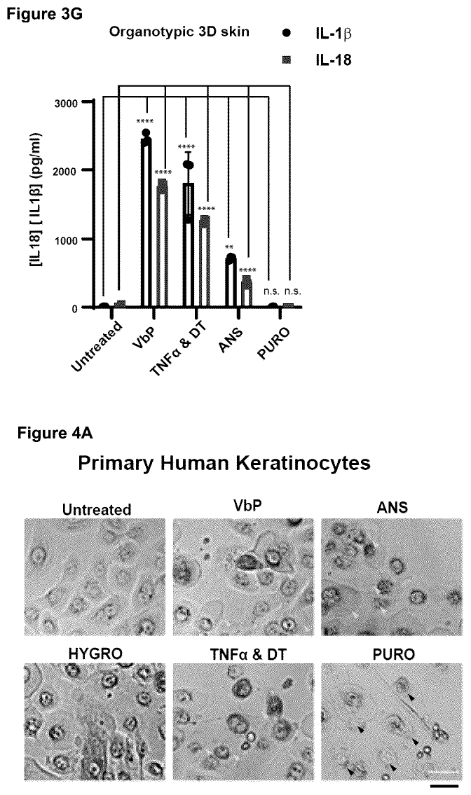

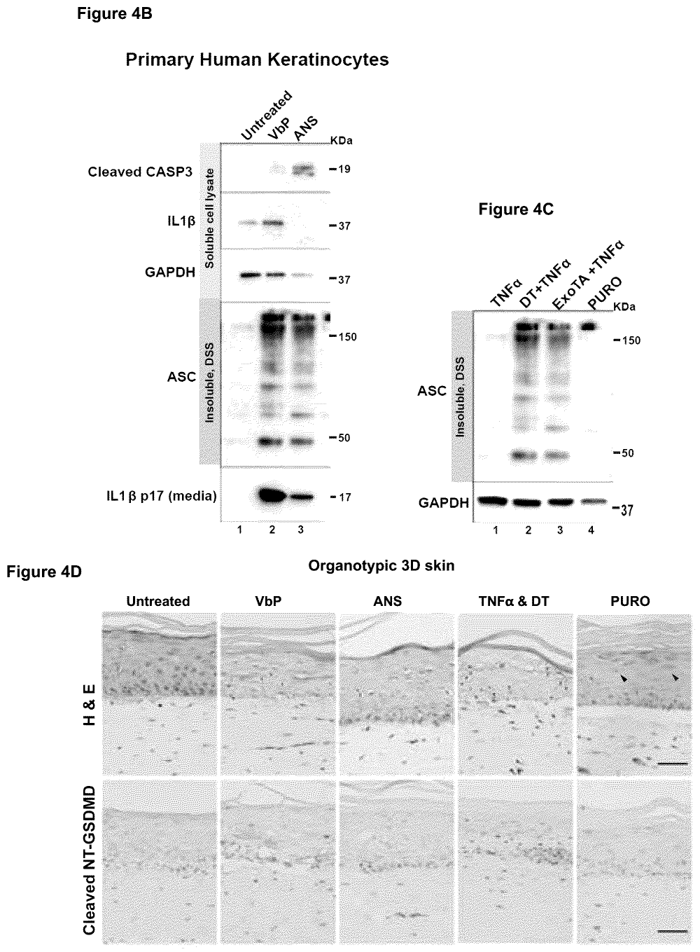

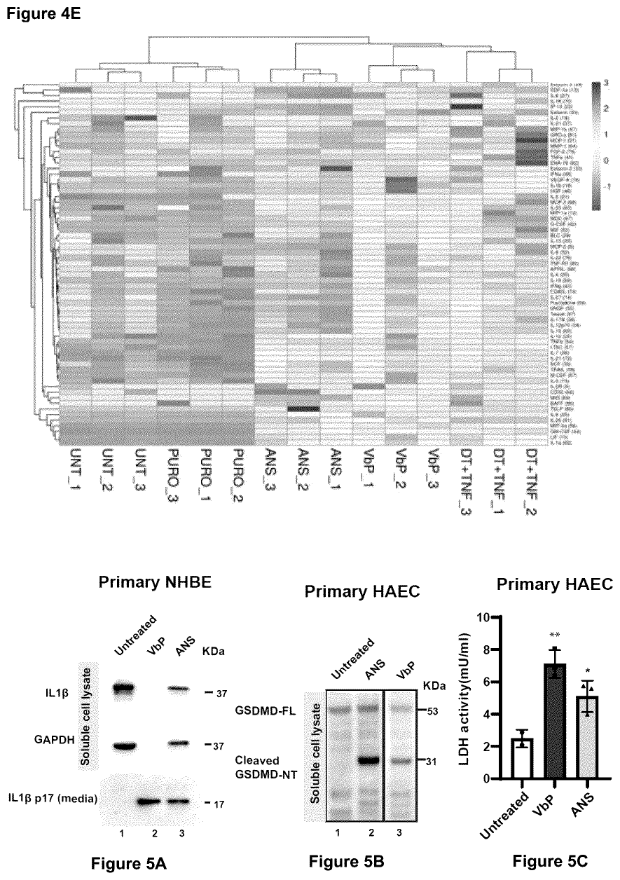

N-TERT keratinocytes were relatively resistant towards DT and ExoTA but can be sensitized by prior priming with TNFα [Mizutani et al., Urological Research 22:261-266 (1994)]. In the case of DT, this is partially explained by increased expression of the DT entry receptor HB-EGF (>5-fold increase, FKPM by RNAseq). Similar to ANS, HYGRO and LTM, TNFα+DT and TNFα+ExoTA elicited all morphological and biochemical hallmarks of pyroptotic cell death in N-TERT cells, including membrane ‘ballooning’, ASC polymerization and the secretion of IL1β p17 (FIGS. 3B and D). Combined Annexin V and PI staining and the appearance of cleaved PARP1 and caspase-3 revealed that DT and ExoTA also triggered apoptosis in TNFα primed N-TERT cells (FIGS. 3C and D). The ability of TNFα+DT, TNFα+ExoTA and ANS to induce inflammasome-driven pyroptosis was also confirmed in primary human keratinocytes freshly isolated from a healthy donor (FIG. 4A-C), indicating that it is not an artifact caused by immortalization.

Both Corynebacterium diphtheriae and Pseudomonas aeruginosa are specialized in colonizing human epithelia, including the skin. In the case of Corynebacterium, DT plays a major role in tissue damage during infection, as the presence of the phage-encoded DT gene alone can distinguish between pathogenic and benign commensal strains (Institute and National Cancer Institute, 2020). Therefore, we tested if DT could cause pyroptosis in fully stratified 3D human organotypic skin. Similar to the NLRP1 agonist VbP, TNFα+DT and ANS treatment caused striking epidermal dyskeratosis, i.e. abnormal keratinocytes with apparent cytosolic vacuolization and condensed, hematoxylin-rich nuclei (FIGS. 3E and 4D (white arrows)). Immunostaining revealed that the pore-forming cleaved GSDMD N-terminal fragment (GSDMD) was prominently enriched at the cell membrane in VbP, ANS and TNFβ+DT treated 3D skin cultures, especially around areas of dyskeratosis (FIG. 3E, insets). These features were absent from untreated 3D skin cultures and differed remarkably from those caused by PURO (FIG. 4D).

In addition, principal analysis and hierarchical clustering clearly distinguished the chemokine/cytokine profiles induced by VbP, ANS and TNFα+DT, with VbP and TNFα+DT being the most similar (FIGS. 3F and 4E). Notably, very few cytokines/chemokines differed between PURO-treated and untreated samples. ELISA confirmed that inflammasome-dependent cytokines IL-1β and IL-18 were among the most enriched in VbP, ANS and TNFα+DT treated samples, with >200-fold induction relative to untreated and PURO-treated samples (FIG. 3G), further proving that ANS and TNFα+DT, similar to VbP, can trigger inflammasome-driven pyroptosis. This observation was not restricted to human skin keratinocytes, as primary bronchial epithelial cells (NHBEs) and aortic endothelial cells (HAECs) also underwent canonical inflammasome activation and pyroptosis (FIG. 5A-C) in response to ANS. Taken together, these results demonstrate that inflammasome-driven pyroptosis is a specific cell-intrinsic immune response against small molecule and proteinaceous ribotoxins that inhibit the elongation phase of the ribosome.

### Example 4

### Human NLRP1, but not NLRP3 or CARDS, Drives Pyroptosis in Response to Ribosome Stalling/Collisions

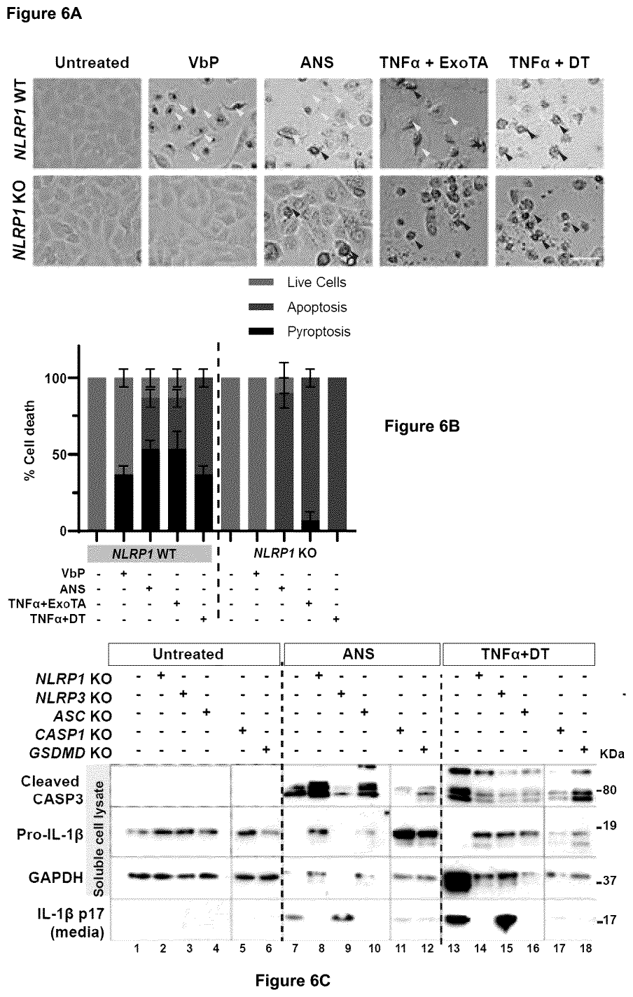

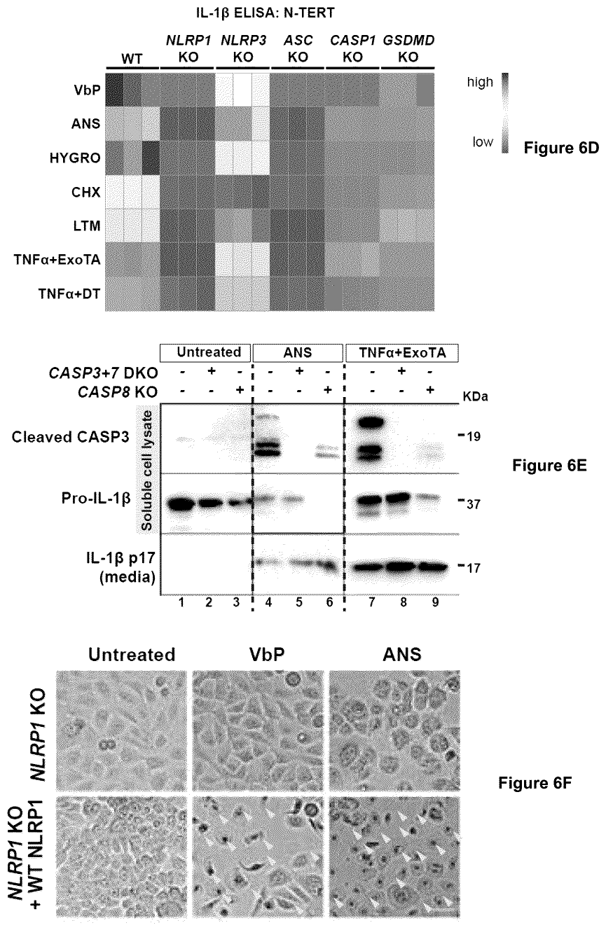

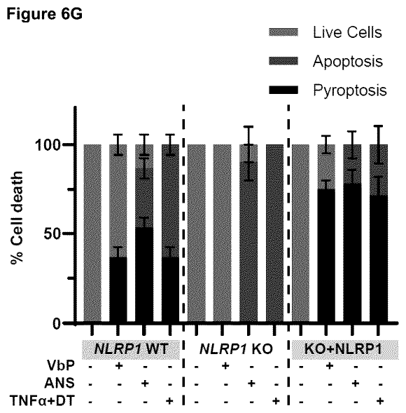

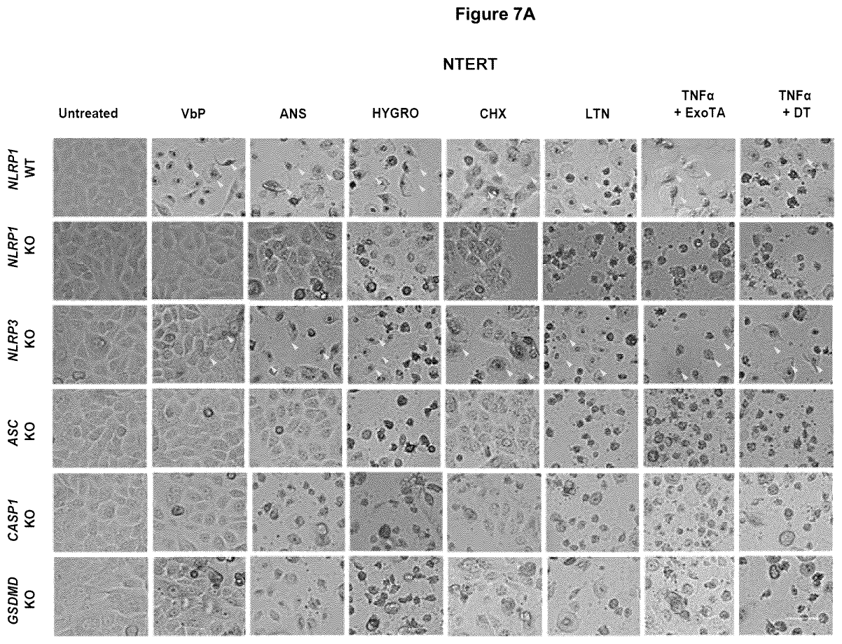

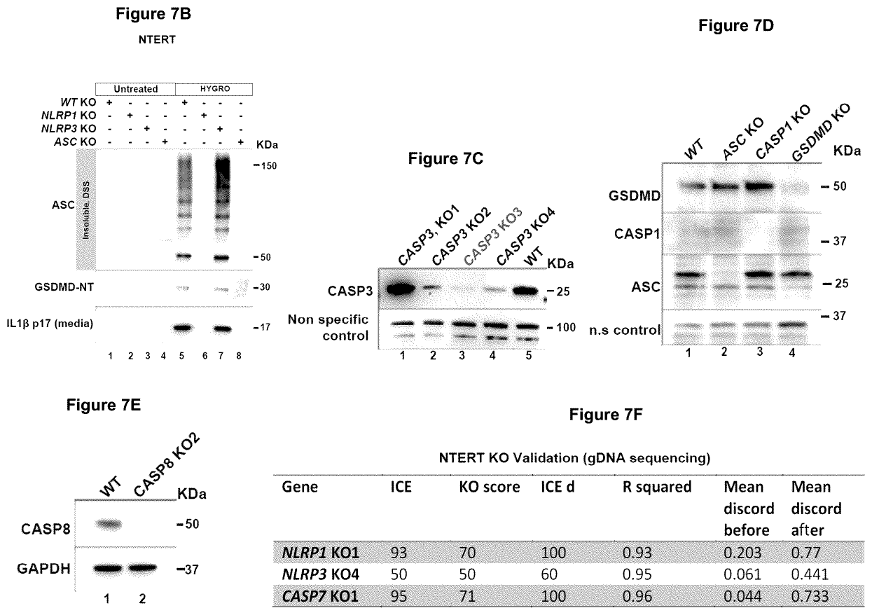

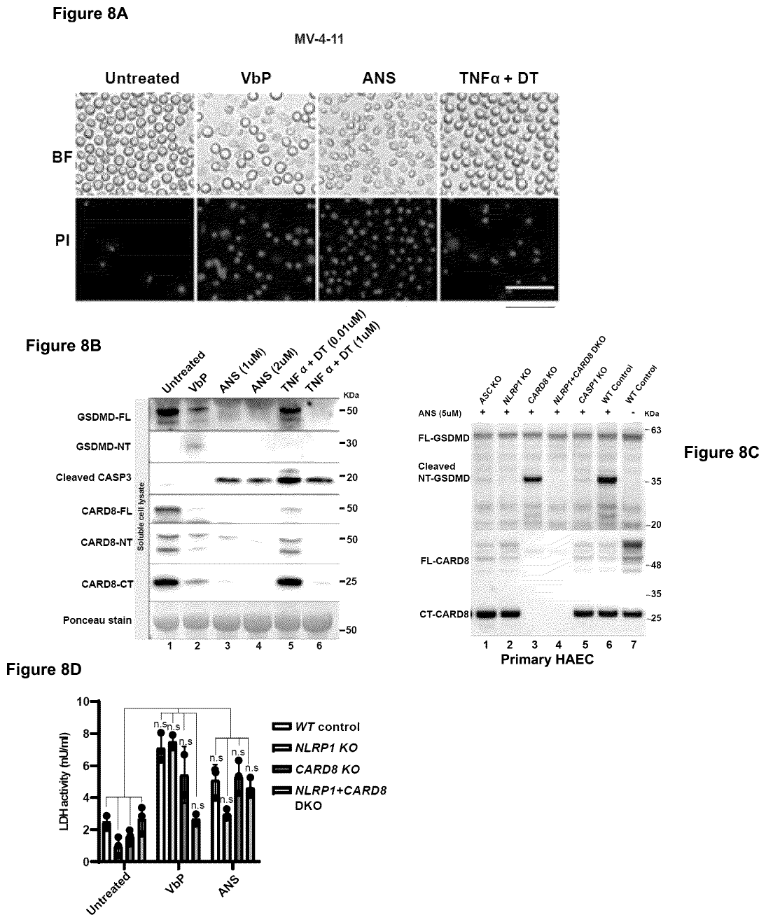

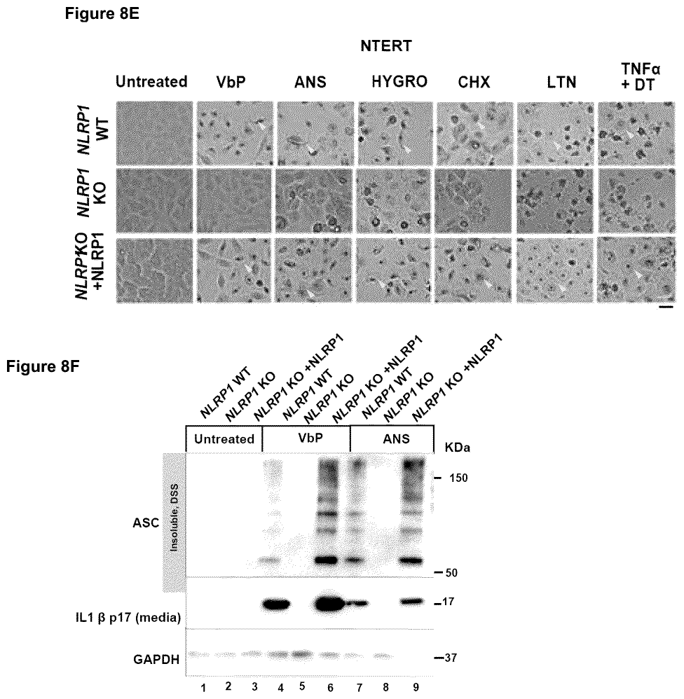

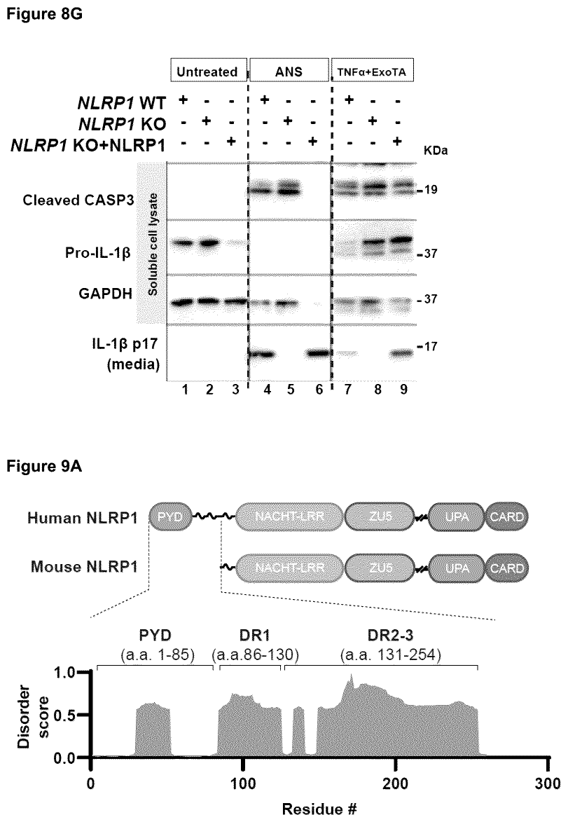

To validate NLRP1 is the responsible sensor for ribosome-targeting chemicals and toxins, we compared Cas9 control N-TERT cells to a panel of polyclonal inflammasome knock-out (KO) cells. Genetic deletion of either NLRP1 (NLRP1 KO) or any of the downstream inflammasome components including ASC, pro-caspase-1 or GSDMD abrogated the characteristic ‘membrane ballooning’ caused by ANS, LTM, HYGRO, TNFα+DT and TNFα+ExoTA (FIG. 6A, FIG. 7A, 7D-F) and caused the intoxicated cells to appear shrivelled, with membrane ‘blebbing’ typically associated with late-stage apoptosis and necrosis (FIGS. 6A and 7A). Notably, the secretion of IL1β p17 in these KOs was also markedly reduced (FIG. 7B, FIG. 6C-D). By contrast, deletion of NLRP3 did not affect either apoptosis or pyroptosis following ANS or TNFα+DT treatment (FIGS. 7A and 6C-D). Combined Annexin V and PI staining confirmed that NLRP1 KO cells treated with ANS, TNFα+DT or TNFα+ExoTA died exclusively through apoptosis (FIG. 6B). Conversely, deletion of pro-apoptotic caspases, caspase-3, 7 and caspase-8 did not diminish the secretion of mature IL1β p17 in N-TERT cells (FIGS. 6E and 7C-D), confirming that ANS- and TNFα+DT-triggered pyroptosis was entirely driven by caspase-1. In primary human aortic endothelial cells (HAECs), NLRP1 deletion abrogated GSDMD cleavage, whereas CARD8 deletion did not have an effect (FIG. 8A-B). A leukemia cell line, MV4-11, which exclusively activates CARD8 instead of NLRP1 after VbP stimulation (Johnson et al., 2018) also did not display detectable GSDMD cleavage after ANS treatment (FIGS. 8A-B). Thus, NLRP3 or CARD8 does not play a major role in sensing ribosome stalling/collisions, at least in the cell types tested. Furthermore, NLRP1 KO N-TERT cells rescued with exogenously expressed wild-type NLRP1 fully regained the ability to undergo cell ‘ballooning’ (FIG. 6F, 8C), assemble ASC oligomers (FIG. 8D), secrete mature IL1β (FIG. 8E) and execute pyroptosis (FIG. 6G) in response to ANS and TNFα+DT. In conjunction with results obtained with 293T-ASC-GFP-NLRP1 reporter cells (FIG. 1), these findings show that endogenous NLRP1 is the necessary and sufficient sensor for all tested pyroptosis-inducing ribosome inhibitors.

### Example 5

### Ribotoxin-Induced NLRP1 Activation Requires ZAKα Kinase and a Human Specific Disordered Region

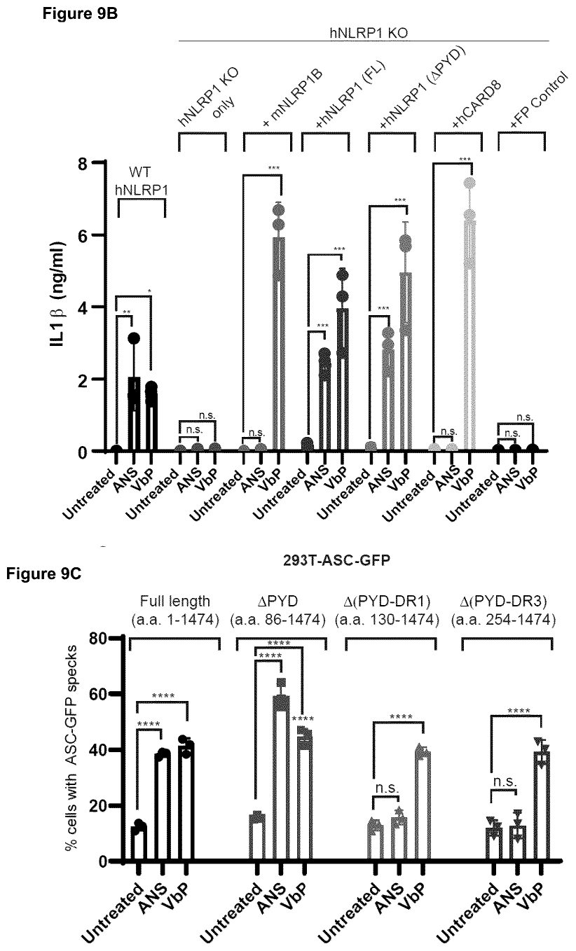

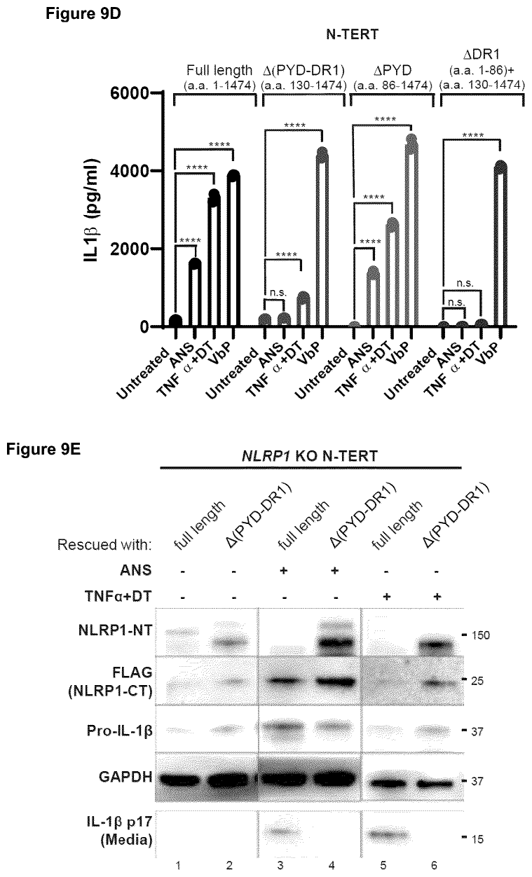

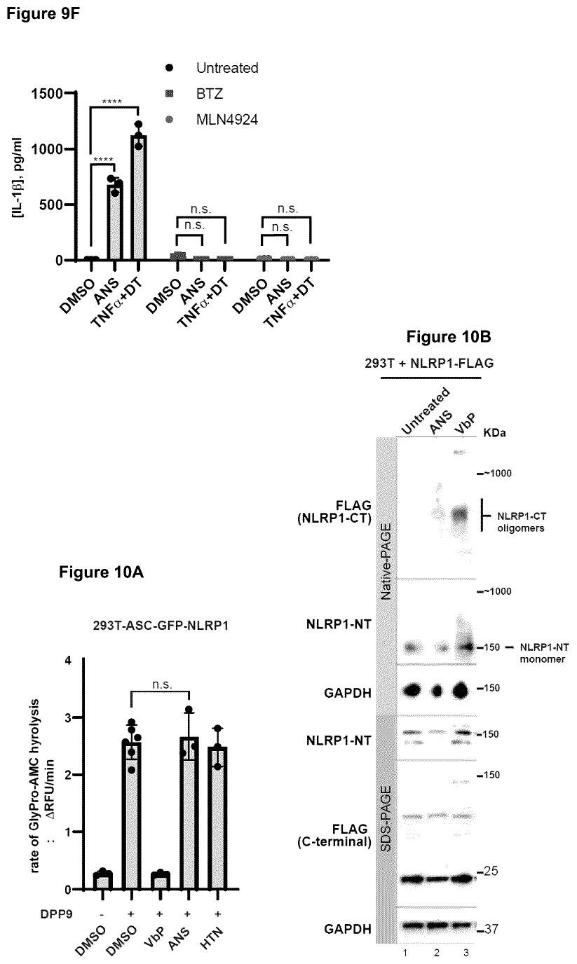

Three classes of human NLRP1 agonists have been identified so far: 1) small molecule inhibitors of cytosolic dipeptidases DPP8 and DPP9 such as VbP [Gai et al., cell Death Disease 10 (2019); Okondo et al., Cell Chem Biol 25:262-267.e5 (2018); Zhong et al., J Biol Chem 293:18864-18878 (2018)] 2) enteroviral 3C proteases (3Cpros) [Robinson et al., Science (2020); Tsu et al., Elife 10 (2021)] and 3) long double-stranded RNAs [Bauernfried et al., Science (2020)]. These agonists activate human NLRP1 via different domains within the NLRP1 N-terminal fragment (NLRP1-NT) (FIG. 9A). In an in vitro proteolysis assay, ANS had no effect on the catalytic activity of human DPP9, in contrast to VbP (FIG. 10A). We also did not observe any smaller bands in ANS-treated cells that would indicate specific cleavage of overexpressed NLRP1, which had been prominently observed in 3Cpro-expressing or HRV-infected cells (FIG. 9B). Since ribotoxins such as ANS are not known to induce dsRNA formation, we postulate that stalled/collided ribosomes activate NLRP1 through a novel pathway not shared with known NLRP1 agonists. To elucidate this biochemically, we took advantage of the observation that only overexpressed human NLRP1, but not CARD8 or murine NLRP1B, could restore ANS-triggered IL1β secretion in NLRP1 KO keratinocytes, even though all three inflammasome sensors could restore VbP-dependent IL-1β secretion (FIG. 10C). Moreover, the level of ANS-triggered IL1β secretion and cell death did not differ between wild-type and NIrp1a/b KO mouse bone marrow derived macrophages (BMDMs) (FIG. 10E). Thus, the ability to sense stalled/collided ribosomes might rely on a unique structural feature of human NLRP1 that is absent in CARD8 and murine NLRP1A/B.

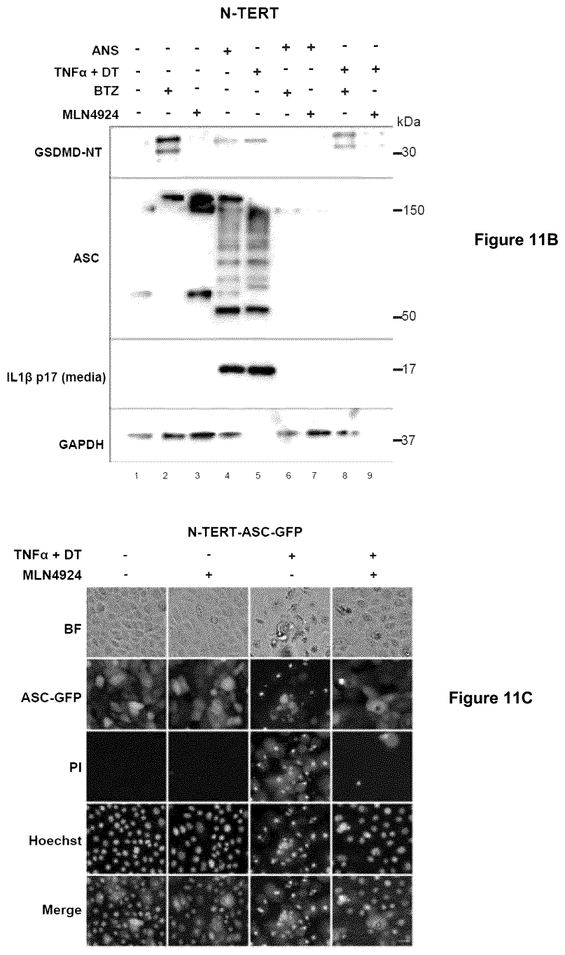

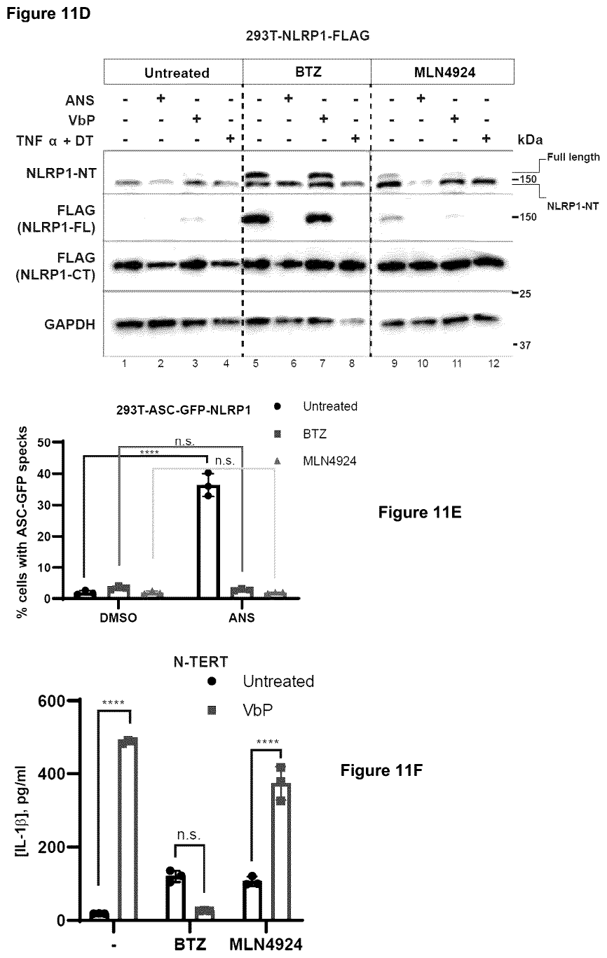

Human NLRP1 harbors a unique N-terminal extension consisting of an atypical pyrin domain (PYD) followed by three predicted disordered regions (DR1: a.a.86-130, DR2: a.a.131-149, DR3: a.a. 150-254) (FIG. 9A). Using a series of N-terminal truncation constructs, we found that the PYD is not required for ANS- or VbP-dependent NLRP1 activation and might in fact limit the effect of ANS (FIG. 9B). By contrast, any deletion that removed the 45-amino acid long DR1 selectively abrogated ANS-triggered ASC-GFP specks formation in 293T-NLRP1-ASC-GFP reporter cells, or ANS/DT-induced IL1β secretion in N-TERT cells (FIGS. 9B and 10D-F), without affecting VbP-triggered NLRP1 activation (FIGS. 9C-D). These results suggest that the human NLRP1 has gained a novel function during evolution to sense stalled/collided ribosomes by the use of a unique N-terminal extension encompassing PYD-DR1. Although it is not involved in sensing VbP, the role of NLRP1-DR1 is reminiscent of a recent study demonstrating a definitive requirement for a N-terminal disordered region during VbP-triggered CARD8 activation [Chui et al., Cell Rep 33:108264 (2020)]. In that context, the CARD8 DR mediates the ‘functional degradation’ of the auto-inhibitory CARD8-NT in response to VbP [Chui et al., Cell Rep 33:108264 (2020)]. To test whether NLRP1-DR1 plays an analogous role in response to ANS, we tracked the fate of the wild-type and Δ(PYD-DR1) NLRP1-NT in NLRP1 KO keratinocytes rescued with full-length NLRP1 or Δ(PYD-DR1) truncation mutant (NLRP1^{ΔPYD-DR1}, a.a.131-1474). ANS and TNFα+DT both caused a significant reduction in the level of intact NLRP1-NT, but not in the Δ(PYD-DR1) mutant (FIG. 9E). By contrast, NLRP1-CT remained unaffected by ANS or TNFα+DT. Thus, similar to other NLRP1 agonists, ribotoxin-triggered NLRP1 activation also proceeds via NLRP1-NT destabilization. Furthermore, we found that this could be blocked by cullin complex inhibitor MLN4924, or proteasome inhibitor bortezomib (FIG. 11D). Both inhibitors also abrogated ANS- and TNFα+DT-triggered NLRP1 activation in 293T-NLRP1-ASC-GFP reporter cells and N-TERTs (FIGS. 11A-F and 9F), confirming that NLRP1-NT degradation via the cullin complex and the proteasome is essential in the subsequent inflammasome activation in response to select ribosome inhibitors.

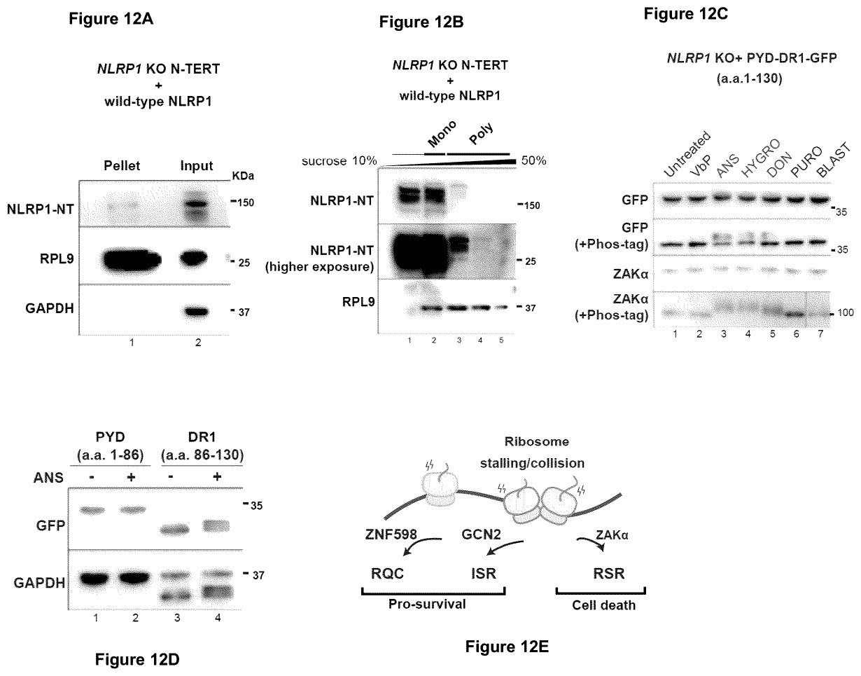

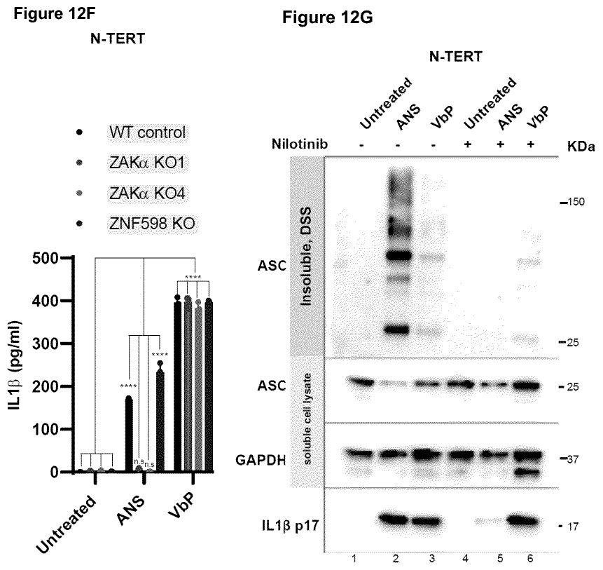

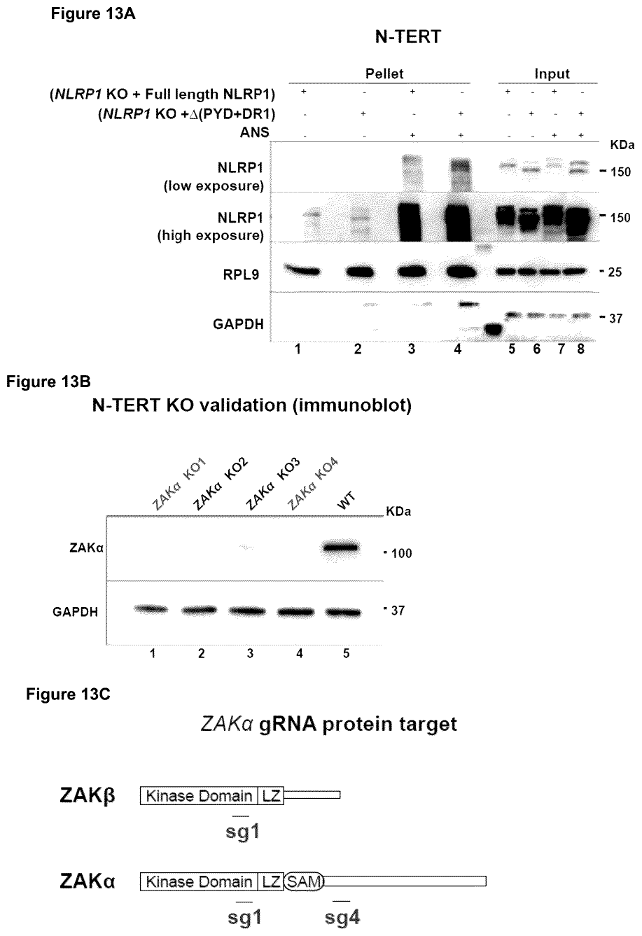

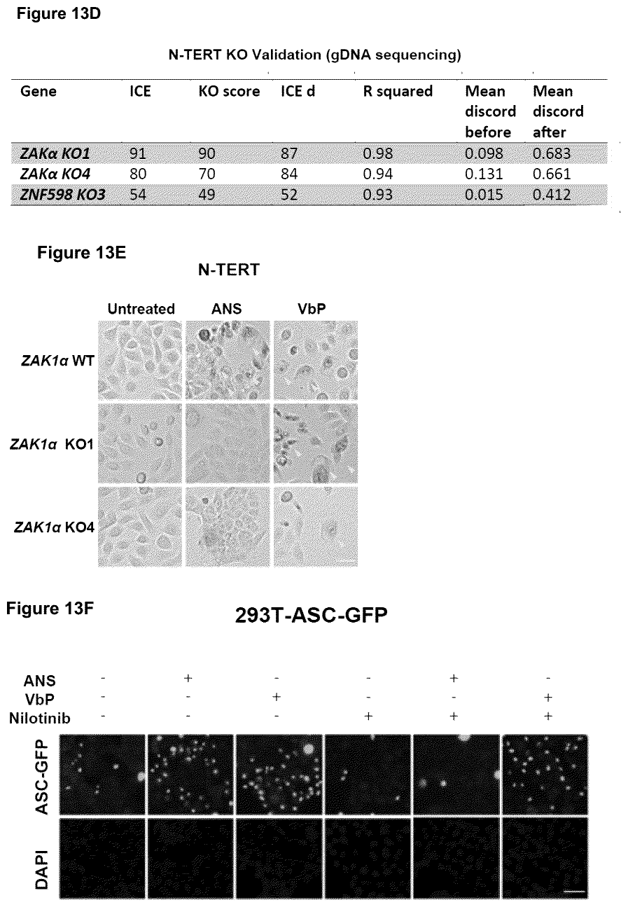

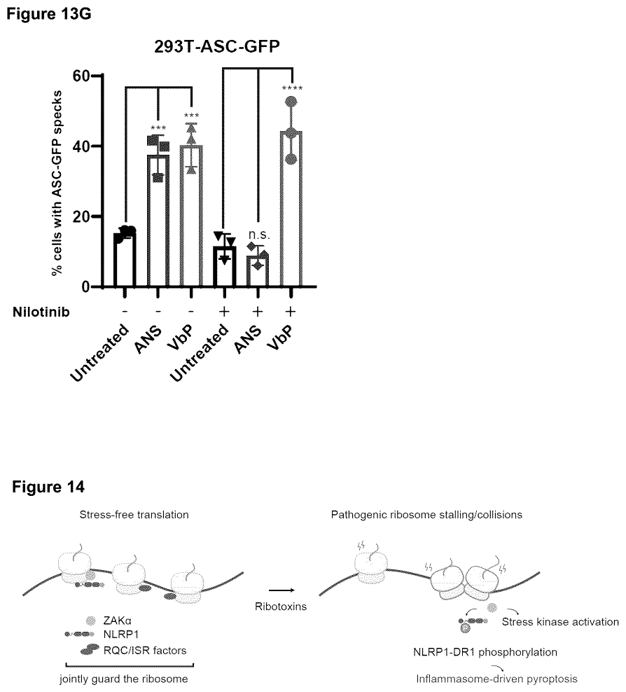

Next the inventors tested whether NLRP1 could sense stalled/collided ribosomes in situ by directly associating with the ribosome complex. A significant amount of NLRP1 co-purified with large ribosome subunit RPL9 through a discontinuous sucrose cushion in N-TERT cell lysates, in contrast to the abundant non-ribosomal protein GAPDH (FIG. 12A). Furthermore, NLRP1 was associated with both monosomes and elongating polysomes through a continuous sucrose gradient (FIG. 12B). Remarkably, ANS treatment dramatically increased the level of ribosome-bound NLRP1, even though its overall level was lower following ANS treatment as a result of NLRP1-NT degradation (FIG. 13A). Thus, NLRP1 is a ribosome-bound immune sensor that is primed to detect ribosome stalling/collisions.

Despite the necessity for DR1 for NLRP1 to sense ribotoxins such as ANS and DT, the recruitment of the ΔPYD-DR1 mutant was not impaired as compared to wild-type full length NLRP1 (FIG. 13A). Thus, DR1 controls a step of NLRP1 activation that is separate from ribosome association. This result also proves that increased recruitment to stalled/collided ribosomes in itself is insufficient, and must require additional signals to activate NLRP1. In order to dissect the role of DR1, we expressed GFP-tagged PYD-DR1 as a separately polypeptide in NLRP1 KO N-TERT cells. While monitoring its expression following ribotoxin treatment, we noticed a band shift in PYD-DR1-GFP in all cells treated with pyroptosis-inducing ribosome inhibitors, including ANS, HYGRO and DON, but not with non-NLRP1 activating ones, such as PURO and BLAST (FIG. 12C). Phos-tag SDS-PAGE identified the slower migrating band to be phosphorylated PYD-DR1-GFP (FIG. 12C). This observation raised the interesting possibility that NLRP1 activation was under the control of a phosphorylation switch by a kinase.

Recently the long isoform of the MAPKKK gene product (ZAKα kinase herein) was found to be a proximal sensor for stalled/collided ribosomes and a master regulator of the downstream ribotoxic stress response (RSR) (FIG. 12D). Constitutively associated with the ribosomes via its C-terminal sensor domains, ZAKα auto-phosphorylates upon ribosome stalling/collisions and rapidly activates stress-responsive kinases such as JNK and p38 [Vind et al., Mol Cell 78 700-713.e7 (2020); Wu et al., Cell 182-404-416.e14 (2020)]. Using auto-phosphorylation as a surrogacy for its activation status, ZAKα shares remarkably similar reactivities towards different ribosome inhibitors as NLRP1 [Vind et al., Mol Cell 78:700-713.e7 (2020)] (FIG. 12C). We therefore postulated that ZAKα functions upstream of NLRP1 PYD-DR1 phosphorylation and NLRP1 activation. Indeed, ZAKα KO N-TERT cells became completely resistant towards ANS-induced IL1β secretion and pyroptotic death but remained fully sensitive to VbP (FIGS. 12E and 13B-E). Deletion of the RQC regulator ZNF598 did not affect NLRP1 activation (FIG. 12E). To confirm that the kinase activity of ZAKα is required, wild-type N-TERT cells and 293T-ASC-GFP-NLRP1 reporter cells were treated with Nilotinib, an FDA approved leukemia drug that has nanomolar IC₅₀ against ZAKα. Nilotinib, similar to ZAKα deletion, abrogated all measures of ANS- and DT-dependent inflammasome activation, including ASC oligomerization and IL1β secretion in N-TERTs (FIG. 12F), as well as ASC-GFP speck formation in 293T-ASC-GFP-NLRP1 reporter cells (FIG. 13F-G), but had no discernible effect on VbP-triggered pyroptosis. Furthermore, suppression of NLRP1 activation by Nilotinib correlated with the loss of NLRP1 PYD-DR1 phosphorylation (FIG. 12G). Thus, the kinase activity of ZAKα is an obligate upstream regulator of NLRP1 activation by stalled/collided ribosomes.

### Example 6

### UVB Induces NLRP1 Via ZAKα

ZAKα senses aberrant ribosomes that have stalled and/or collided after encountering a translocation-blocking mRNA lesion, such as those induced by UVB. Activated ZAKα undergoes extensive self-phosphorylation and phosphorylates downstream SAPKs such as p38 and JNK. Collectively, this pathway was termed the ribotoxic stress response (RSR). Due to its shared involvement for RNA damage, we examined whether RSR intersects with UVB-induced NLRP1 activation.

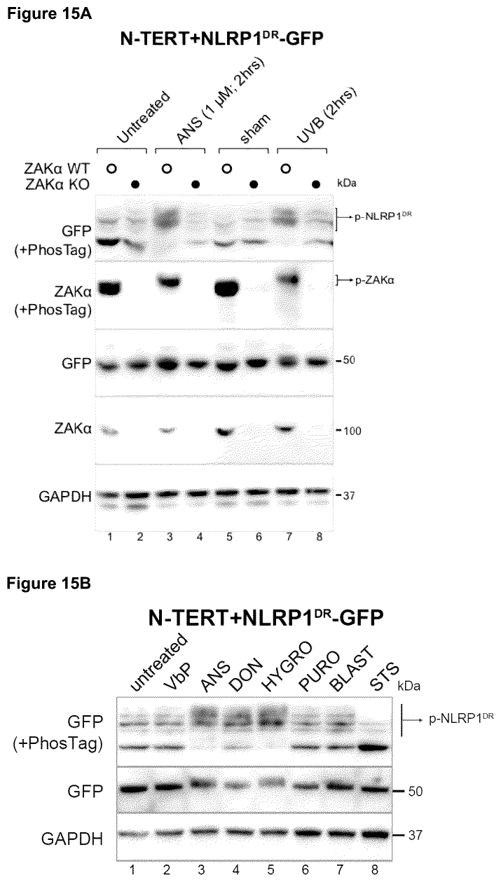

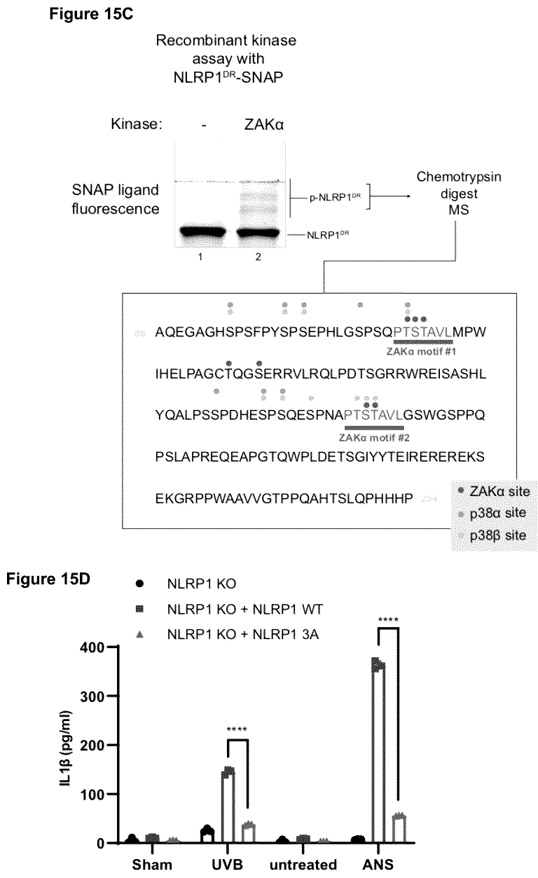

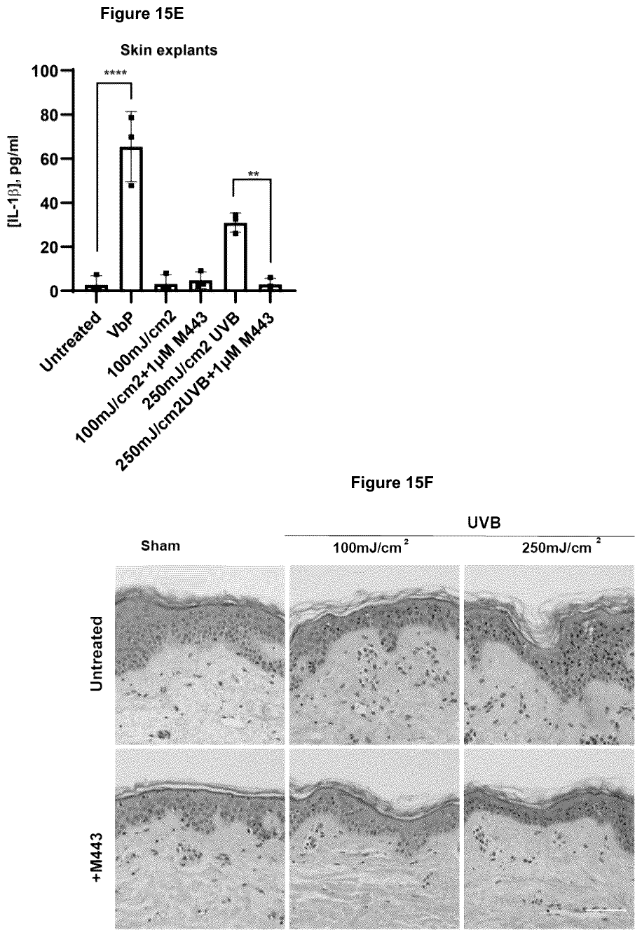

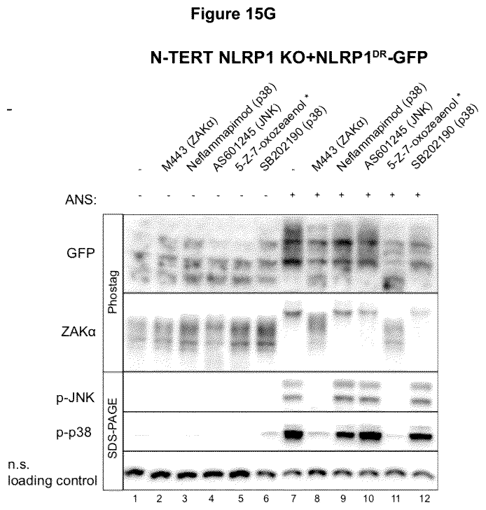

A reduction in UVB-induced IL1β was observed in human skin explants treated with a specific ZAKα inhibitor M443; IUPAC name 4-methyl-N-[3-(4-methylimidazol-1-yl)-5-(trifluoromethyl)phenyl]-3-[[4-(1-prop-2-enoylpiperidin-3-yl)pyrimidin-2-yl]amino]benzamide (CAS No. 1820684-31-8) (FIG. 15E-F). ZAKα KO or deletion did not affect VbP-driven pyroptosis in N-TERT cells. Thus, ZAKα is selectively required for the NLRP1 inflammasome activation downstream of UVB.

### UVB-Induced ZAKα Phosphorylation of NLRP1^{DR}

Given the requirement of the NLRP1^{DR} in response to UVB and ANS, we examined the behavior of NLRP1^{DR} as a fusion protein with GFP. A marked band shift for NLRP1DR-GFP was observed by immunoblot whenever cells were treated with UVB or ANS; whereas in ZAKα KO N-TERT cells, this bandshift was completely abrogated. Phostag gel confirmed that the band shift was due to NLRP1^{DR} phosphorylation. A basal level of NLRP1^{DR} phosphorylation remained unaffected by ANS or UVB (FIG. 15A). This observed band shift was sensitive to post-lysis treatment with lambda phosphatase (LPP) and could be eliminated by mutating all the serine and threonine residues to alanine (This mutant is hereby referred to as “STless”), confirming that it was caused by phosphorylated NLRP1DR. PhosTag SDS-PAGE further revealed that NLRP1 DR was in fact significantly phosphorylated in unstimulated cells, and became further phosphorylated by ANS and UVB (FIG. 15A) with almost no unphosphorylated species left 2 hours post treatment. We hereby refer to ANS- or UVB-dependent NLRP1^{DR} phosphorylation as ‘hyperphosphorylation’. Importantly, other ZAKα-activating drugs, such as HYGRO and DON, also led to NLRP1^{DR} hyperphosphorylation, but not VbP or any of the non-ZAKα-activating cytotoxic compounds (FIG. 15B).

In ZAKα KO N-TERT cells, ANS- and UVB-induced NLRP1^{DR} ‘hyperphosphorylation’ was completely abrogated, while NLRP1^{DR} basal phosphorylation remained unaffected (FIG. 15A). The inflammasome adaptor ASC is not phosphorylated by either ANS or VbP treatment (data not shown), Thus ZAKα-driven NLRP1 phosphorylation is specific. In orthogonal experiments, co-expression of wild-type ZAKα induced a large bandshift of full length NLRP1 in 293T cells, which was strongly diminished by the deletion of NLRP1^{DR}, the ZAKα kinase dead mutation (p.K45A) or the removal of ZAKα RSR sensing domains (CTD and S domains) (data not shown). Thus, ZAKα, when activated either by overexpression or ribotoxic stress, hyperphosphorylates full length NLRP1 within the disordered linker region.

### Example 7

ZAKα Phosphorylates a PTSTAVL Motif within NLRP1^{DR}

Mutating the serine/threonine residues within a short stretch of NLRP1^{DR} (a.a. 121-196) to alanine abrogated NLRP1 activation by UVB, but did not affect VbP-driven IL1β secretion. This suggests that the a.a. 121-196 harbors functionally critical ZAKα dependent phosphorylation sites, although additional phosphorylation sites might exist. Importantly recombinant ZAKα is sufficient to phosphorylate SNAP-tagged NLRP1^{DR} purified from bacteria (FIG. 15C), suggesting that NLRP1 is a direct substrate of ZAKα. Mass spectrometry of excised p-NLRP1^{DR} bands after co-incubation with ZAKα uncovered 7 distinct ZAKα phosphorylation sites. Remarkably, these sites cluster in two identical motifs of the sequence ‘PTSTAVL’ (SEQ ID NO: 16) (FIG. 15C, box), which does not exist in any other protein in the human proteome (Uniprot BLAST search). Hence, the inventors propose to name this sequence ‘ZAKα motif’. Mutating the three serine/threonine residues within this motif (a.a. T178A, S179A, T180A, designated as NLRP1 ‘3A’ mutant) eliminated UVB- and ANS-induced pyroptosis in reconstituted NLRP1 KO N-TERT cells, but had no effect on VbP-dependent pyroptosis (FIG. 15D). The results identified a single phosphorylation site in NLRP1^{DR} that is indispensable for ZAKα-driven NLRP1 inflammasome activation.

FIG. 15G shows that both p38α/β can phosphorylate NLRP1^{DR} in a recombinant kinase assay, including the same residues within the ZAKα hyperphosphorylation in ANS-treated N-TERT cells. This was in contrast to ZAKα inhibitors, which completely abrogated NLRP1 DR hyperphosphorylation (FIG. 15G).

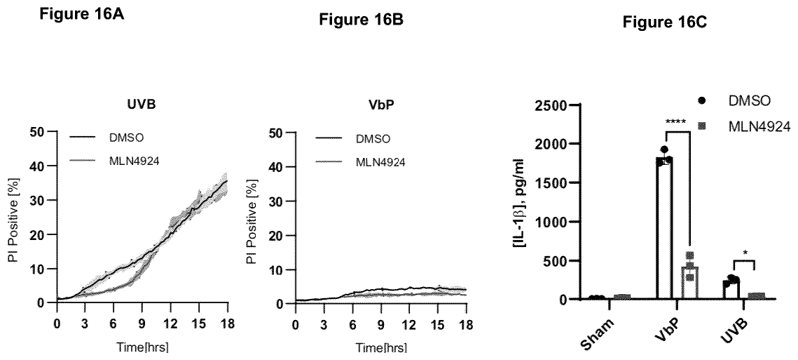

The inventors have further observed that UVB-dependent NLRP1 activation is accompanied by a decrease in NLRP1 N-terminal fragment (NT) and is abrogated by the NEDD8/cullin inhibitor MLN4924 (FIG. 16A-C).

### SUMMARY

Based on these results, the inventors propose that human NLRP1 functions as a specific inflammasome sensor for ‘ribotoxic stress’ caused by stalled/collided ribosomes (FIG. 14). At the basal state, a fraction of NLRP1, along with its upstream regulator ZAKα, is constitutively associated with the ribosome in a state of immune surveillance. While sporadically stalled ribosomes can be repaired through the RQC pathway, an abnormally high level of ribosome stalling and/or collisions could be a telltale sign of pathogen attack and thus triggers ZAKα-dependent RSR. ZAKα, or a downstream kinase, then phosphorylates the ‘PTSTAVL’ motif in the human specific disordered linker region of NLRP1 and leads to the cullin-mediated degradation of the entire auto-inhibitory NLRP1-NT. This sequence of events culminates in NLRP1-CT-driven assembly of the inflammasome complex and commits the infected cell to pyroptosis.

In this work, the key events controlling UVB-triggered NLRP1 inflammasome activation in keratinocytes were resolved. By inducing cellular RNA photo-lesions that stall the ribosomes, UVB activates the ribotoxic response (RSR) kinase ZAKα, which, together with its downstream effectors p38, directly phosphorylates the human specific disordered linker region of NLRP1. A single phosphorylation site within the ZAKα motif identified here is sufficient to act as ‘ON’ switch for NLRP1. Notably, the ZAKα dependent mechanism of activation is entirely uncoupled from DPP8/9.

The inventors' results also challenge the long-held dogma that translation inhibitors are non-specific cytotoxic agents that result purely in apoptosis. This discrepancy is most likely due to the fact that NLRP1 is silenced in nearly all human cancer cell lines of epithelial origin. As certain ribosome inhibitors have been used successfully as cancer drugs and antivirals [Reuschl et al., bioRxiv (2021); Shafiee et al., Front Microbiol 10:2340 (2019); White et al., Science (2021)], it is conceivable that NLRP1-driven pyroptosis might contribute to the therapeutic response of these drugs in patients.

Along with recent discoveries of other NLRP1 triggers, our findings highlight the remarkable versatility of NLRP1 as an immune sensor. Through its modular domains, NLRP1 can either bind pathogen-associated pattern molecules (PAMP) directly (dsRNA), respond to viral enzymes (3Cpro) via substrate mimicry, or sense unusual changes of a key cellular process, i.e. translation of mRNA by the ribosome. In the latter case, human NLRP1 functions analogously to plant ‘guard’-type immune sensors that monitor certain cellular proteins which are especially vulnerable to pathogen attack [Jones et al., Science 354 (2016)]. As ribosome-targeting toxins are very common among microbial pathogens, this ‘guard’ (NLRP1)-‘guardee’ (elongating ribosome) relationship, which is absent in most non-primate species, might have evolved as part of the arms race to cope with microbial pathogens that are particularly virulent for primates. In addition, pharmacologic inhibition of the ZAKα-NLRP1 axis might provide a useful therapeutic strategy in treating human inflammatory disorders.

### REFERENCES

1. Arnette, C., et al., (2016). In Vitro Model of the Epidermis: Connecting Protein Function to 3D Structure. Methods Enzymol. 569, 287-308.
2. Bauernfeind, F., and Hornung, V. (2013). Of inflammasomes and pathogens—sensing of microbes by the inflammasome. EMBO Mol. Med. 5, 814-826.
3. Bauernfried, S., et al., (2020). Human NLRP1 is a sensor for double-stranded RNA. Science.
4. Briard, B., et al. (2020). Galactosaminogalactan activates the inflammasome to provide host protection. Nature.
5. Broz, P., and Dixit, V. M. (2016). Inflammasomes: mechanism of assembly, regulation and signalling. Nat. Rev. Immunol. 16, 407-420.
6. Chen, J., and Chen, Z. J. (2018). Ptdlns4P on dispersed trans-Golgi network mediates NLRP3 inflammasome activation. Nature 564, 71-76.
7. Chui, A. J., et al., (2020). Activation of the CARD8 Inflammasome Requires a Disordered Region. Cell Rep. 33, 108264.
8. Cirelli, K. M., et al., (2014). Inflammasome sensor NLRP1 controls rat macrophage susceptibility to Toxoplasma gondii. PLoS Pathog. 10, e1003927.
9. Dickson, M. A., et al., (2000). Human keratinocytes that express hTERT and also bypass a p16(INK4a)-enforced mechanism that limits life span become immortal yet retain normal growth and differentiation characteristics. Mol. Cell. Biol. 20, 1436-1447.
10. Drutman, S. B., et al. (2019). Homozygous gain-of-function mutation in siblings with a syndromic form of recurrent respiratory papillomatosis. Proc. Natl. Acad. Sci. U.S.A 116, 19055-19063.
11. Endo, Y. (2014). Enzymology of the Ribosome-inactivating Proteins. Ribosome-Inactivating Proteins 151-160.
12. Fenini, G. et al., Genome Editing of Human Primary Keratinocytes by CRISPR/Cas9 Reveals an Essential Role of the NLRP1 Inflammasome in UVB Sensing. J Invest Dermatol 138:2644-2652 (2018).
13. Gai, K., et al., (2019). DPP8/9 inhibitors are universal activators of functional NLRP1 alleles. Cell Death & Disease 10.
14. Gao, W., et al., (2016). Site-specific phosphorylation and microtubule dynamics control Pyrin inflammasome activation. Proc. Natl. Acad. Sci. U.S.A 113, E4857-E4866.
15. Garreau de Loubresse, N., et al., (2014). Structural basis for the inhibition of the eukaryotic ribosome. Nature 513, 517-522.
16. Grandemange, S., et al. (2017). A new autoinflammatory and autoimmune syndrome associated with NLRP1 mutations: NAIAD (NLRP1-associated autoinflammation with arthritis and dyskeratosis). Ann. Rheum. Dis. 76, 1191-1198.
17. Institute, N.C., and National Cancer Institute (2020). Corynebacterium diphtheriae. Definitions.
18. Jiménez, A., and Vázquez, D. (1979). Anisomycin and Related Antibiotics. Mechanism of Action of Antieukaryotic and Antiviral Compounds 1-19.
19. Johnson, D. C., et al. (2018). DPP8/DPP9 inhibitor-induced pyroptosis for treatment of acute myeloid leukemia. Nat. Med. 24, 1151-1156.
20. Jones, J. D. G., Vance, R. E., and Dangl, J. L. (2016). Intracellular innate immune surveillance devices in plants and animals. Science 354.
21. Joost, S. et al., Single-Cell Transcriptomics Reveals that Differentiation and Spatial Signatures Shape Epidermal and Hair Follicle Heterogeneity. Cell Syst 3:221-237.e9 (2016).
22. Levinsohn, J. L., et al., (2012). Anthrax lethal factor cleavage of NIrp1 is required for activation of the inflammasome. PLoS Pathog. 8, e1002638.
23. Lopes Fischer, N., et al., (2020). Effector-triggered immunity and pathogen sensing in metazoans. Nat Microbiol 5, 14-26.
24. Macias-Silva, M., Vazquez-Victorio, G., and Hernandez-Damian, J. (2010). Anisomycin is a Multifunctional Drug: More than Just a Tool to Inhibit Protein Synthesis. Current Chemical Biology 4, 124-132.
25. Mitchell, P. S., Sandstrom, A., and Vance, R. E. (2019). The NLRP1 inflammasome: new mechanistic insights and unresolved mysteries. Curr. Opin. Immunol. 60, 37-45.
26. Mizutani, Y., Bonavida, B., and Yoshida, O. (1994). Cytotoxic effect of diphtheria toxin used alone or in combination with other agents on human renal cell carcinoma cell lines. Urological Research 22, 261-266.
27. Mogensen, T. H. (2009). Pathogen Recognition and Inflammatory Signaling in Innate Immune Defenses. Clinical Microbiology Reviews 22, 240-273.
28. Moradali, M. F., et al., (2017). Pseudomonas aeruginosa Lifestyle: A Paradigm for Adaptation, Survival, and Persistence. Frontiers in Cellular and Infection Microbiology 7.
29. Okondo, M. C., et al. (2017). DPP8 and DPP9 inhibition induces pro-caspase-1-dependent monocyte and macrophage pyroptosis. Nat. Chem. Biol. 13, 46-53.
30. Okondo, M. C., et al., (2018). Inhibition of Dpp8/9 Activates the NIrp1b Inflammasome. Cell Chem Biol 25, 262-267.e5.
31. Rathinam, V. A. K., and Fitzgerald, K. A. (2016). Inflammasome Complexes: Emerging Mechanisms and Effector Functions. Cell 165, 792-800.
32. Reuschl, A.-K., et al. (2021). Host-directed therapies against early-lineage SARS-CoV-2 retain efficacy against B.1.1.7 variant. bioRxiv.
33. Robertus, J. D., and Monzingo, A. F. (2014). The Structure and Action of Ribosome-inactivating Proteins. Ribosome-Inactivating Proteins 111-133.
34. Robinson, K. S., et al. (2020). Enteroviral 3C protease activates the human NLRP1 inflammasome in airway epithelia. Science.
35. Sand, J., et al., (2018). Expression of inflammasome proteins and inflammasome activation occurs in human, but not in murine keratinocytes. Cell Death Dis. 9, 24.
36. Schneider-Poetsch, T., et al., (2010). Inhibition of eukaryotic translation elongation by cycloheximide and lactimidomycin. Nat. Chem. Biol. 6, 209-217.
37. Shafiee, F., Aucoin, M. G., and Jahanian-Najafabadi, A. (2019). Targeted Diphtheria Toxin-Based Therapy: A Review Article. Front. Microbiol. 10, 2340.
38. Sharma, N.C., et al., (2019). Diphtheria. Nat Rev Dis Primers 5, 81.
39. Somani, A. K., et al., (2019). Smart Systems and IoT: Innovations in Computing: Proceeding of SSIC 2019 (Springer Nature).
40. Storek, K. M., and Monack, D. M. (2015). Bacterial recognition pathways that lead to inflammasome activation. Immunol. Rev. 265, 112-129.
41. Stuart, L. M., Paquette, N., and Boyer, L. (2013). Effector-triggered versus pattern-triggered immunity: how animals sense pathogens. Nat. Rev. Immunol. 13, 199-206.
42. Sugawara, K., et al., (1992). Lactimidomycin, a new glutarimide group antibiotic. Production, isolation, structure and biological activity. J. Antibiot. 45, 1433-1441.
43. Taabazuing, C. Y., Griswold, A. R., and Bachovchin, D. A. (2020). The NLRP1 and CARD8 inflammasomes. Immunol. Rev. 297, 13-25.
44. Takeuchi, O., and Akira, S. (2010). Pattern Recognition Receptors and Inflammation. Cell 140, 805-820.
45. Tsu, B. V., et al., (2021). Diverse viral proteases activate the NLRP1 inflammasome. Elife 10.
46. Vanaja, S. K., Rathinam, V. A. K., and Fitzgerald, K. A. (2015). Mechanisms of inflammasome activation: recent advances and novel insights. Trends Cell Biol. 25, 308-315.
47. de Vasconcelos, N. M., et al. (2019). DPP8/DPP9 inhibition elicits canonical NIrp1b inflammasome hallmarks in murine macrophages. Life Sci Alliance 2.
48. van de Veerdonk, F. L., et al., (2011). Inflammasome activation and IL1β and IL-18 processing during infection. Trends in Immunology 32, 110-116.
49. Vind, A. C., et al. (2020). ZAKα Recognizes Stalled Ribosomes through Partially Redundant Sensor Domains. Mol. Cell 78, 700-713.e7.
50. Vyleta, M. L., Wong, J., and Magun, B. E. (2012). Suppression of ribosomal function triggers innate immune signaling through activation of the NLRP3 inflammasome. PLoS One 7, e36044.
51. Wang, K., et al. (2020). Structural Mechanism for GSDMD Targeting by Autoprocessed Caspases in Pyroptosis. Cell 180, 941-955.e20.
52. White, K. M., et al. (2021). Plitidepsin has potent preclinical efficacy against SARS-CoV-2 by targeting the host protein eEF1A. Science.
53. Wu, C. C.-C., et al., (2020). Ribosome Collisions Trigger General Stress Responses to Regulate Cell Fate. Cell 182, 404-416.e14.
54. Xu, H., et al. (2014). Innate immune sensing of bacterial modifications of Rho GTPases by the Pyrin inflammasome. Nature 513, 237-241.
55. Zhao, Y., and Shao, F. (2016). Diverse mechanisms for inflammasome sensing of cytosolic bacteria and bacterial virulence. Curr. Opin. Microbiol. 29, 37-42.
56. Zhong, F. L., et al. (2016). Germline NLRP1 Mutations Cause Skin Inflammatory and Cancer Susceptibility Syndromes via Inflammasome Activation. Cell 167, 187-202.e17.
57. Zhong, F. L., et al. (2018). Human DPP9 represses NLRP1 inflammasome and protects against autoinflammatory diseases via both peptidase activity and FIIND domain binding. J. Biol. Chem. 293, 18864-18878.

1. A composition comprising a ZAKα kinase inhibitor and/or a NLRP1 inhibitor for inhibiting NLRP1-driven pyroptosis in a cell caused by ribosome stalling and/or ribosome collisions within said cell.

2. The composition of claim 1, wherein;
a) the ZAKα kinase inhibitor is:
i) Nilotinib, IUPAC name 4-methyl-N-[3-(4-methylimidazol-1-yl)-5-(trifluoromethyl)phenyl]-3-[(4-pyridin-3-ylpyrimidin-2-yl)amino]benzamide,
M443, IUPAC name 4-methyl-N-[3-(4-methyl-1H-imidazol-1-yl)-5-(trifluoromethyl)phenyl]-3-[[4-[1-(1-oxo-2-propen-1-yl)-3-piperidinyl]-2-pyrimidinyl]amino]-benzamide, or
5-Z-7-oxozeanol;
ii) CRISPR-Cas, or
iii) an aptamer; and/or
b) the NLRP1 inhibitor is:
i) MLN4924, IUPAC name ((1S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate, or hydrochloride salt thereof,
TAS4464, IUPAC name 7H-Pyrrolo[2,3-d]pyrimidin-4-amine, 7-[5-[(aminosulfonyl)amino]-5-deoxy-beta-D-ribofuranosyl]-5-[2-(2-ethoxy-6-fluorophenyl)ethynyl]-, or hydrochloride salt thereof;
ZM223, IUPAC name N-[6-[[2-(4-aminophenyl)sulfanylacetyl]amino]-1,3-benzothiazol-2-yl]-4-(trifluoromethyl)benzamide; or
Bortezomib, IUPAC Name: [(1R)-3-methyl-1-[[(2S)-3-phenyl-2-(pyrazine-2-carbonyl amino)propanoyl]amino]butyl]boronic acid; or
ii) CRISPR Cas, or
iii) an aptamer.

3. The composition of claim 1 or 2, wherein the ribosome stalling and/or ribosome collisions are caused by a ZAKα-activating ribotoxin or UVB irradiation.

4. The composition of claim 2 or 3, wherein:
A) the CRISPR-Cas targets (a) ZAKα kinase or (b) NLRP1; or
B) the aptamer targets (a) the kinase domain of ZAKα kinase or (b) one or more ZAKα kinase phosphorylation sites within a sequence motif comprising the amino acid sequence PTSTAVL (SEQ ID NO: 16) of NLRP1.

5. The composition of claim 4, wherein the sequence motif comprises amino acids T178, S179 and T180 of the amino acid sequence of NLRP1 set forth in SEQ ID NO:18.

6. A composition to activate NLRP1-driven pyroptosis in a cell, comprising a compound that causes ribosome stalling and/or ribosome collisions in said cell.

7. The composition of claim 6, wherein the compound activates ZAKα kinase.

8. The composition of claim 7, wherein the compound is a ribotoxin or a ribotoxin conjugated to a targeting molecule such as an antibody.

9. The composition of claim 8, wherein the compound is a ribotoxin selected from the group comprising Anisomycin, Hygromycin, Deoxynivalenol, Diphtheria Toxin and Exotoxin A (from Pseudomonas aeruginosa).

10. Use of a composition according to any one of claims 1 to 5 in the manufacture of a medicament for the treatment of an inflammatory pathology triggered by NLRP1-driven pyroptosis caused by ribosome stalling and/or ribosome collisions.

11. The use according to claim 10, wherein the ribosome stalling and/or ribosome collisions are caused by a ZAKα-activating ribotoxin or UVB irradiation.

12. The use according to claim 11, wherein the inflammatory pathology is due to a microbial ribotoxin.

13. The use according to claim 10, wherein the inflammatory pathology is
i) sunburn caused by UVB irradiation, or
ii) UV-driven skin photosensitivity.

14. The use according to claim 13, wherein the skin photosensitivity is in a subject with lupus erythematosus or bullous pemphigoid and serious solar urticaria

15. Use of a composition according to any one of claims 6 to 9 in the manufacture of a medicament for activating NLRP1-driven pyroptosis.

16. The use according to claim 15, wherein the medicament is for the treatment of cancer.

17. A method of treating an inflammatory pathology triggered by ribosome stalling and/or ribosome collisions, the method comprising administering to a subject in need thereof an efficacious amount of a composition of any one of claims 1 to 5.

18. The method of claim 17, wherein the ribosome stalling and/or ribosome collisions are caused by a ZAKα-activating ribotoxin.

19. The method of claim 18 wherein the ribotoxin is produced by Corynebacterium Diphtheria or Pseudomonas aeruginosa infection, or is a fungal deoxynivalenol toxin.

20. A method of treating a sunburn or skin photosensitivity disorder caused by UVB irradiation, the method comprising administering to a subject in need thereof an efficacious amount of a composition of any one of claims 1 to 5.

21. The method of claim 20 wherein the UVB irradiation is from a solar or an artificial source.


SEQUENCE LISTING (20 sequences)

SEQ ID NO: 1 - DNA, 19 bp, Artificial Sequence
  NLRP1 sgRNA target sequence
gatagcccgagtgcatcgg

SEQ ID NO: 2 - DNA, 20 bp, Artificial Sequence
  ASC sgRNA target sequence
gctaacgtgctgcgcgacat

SEQ ID NO: 3 - DNA, 20 bp, Artificial Sequence
  CASP1 sgRNA target sequence
acagacaagggtgctgaaca

SEQ ID NO: 4 - DNA, 20 bp, Artificial Sequence
  NLRP3 sgRNA target sequence
gaatcccactgtgatatgcc

SEQ ID NO: 5 - DNA, 20 bp, Artificial Sequence
  GSDMD sgRNA target sequence
aggttgacacacttataacg

SEQ ID NO: 6 - DNA, 20 bp, Artificial Sequence
  CASP3 sgRNA target sequence
cgtggtacagaactggactg

SEQ ID NO: 7 - DNA, 20 bp, Artificial Sequence
  CASP7 sgRNA target sequence
ggtacaaacgaggaccggtc

SEQ ID NO: 8 - DNA, 20 bp, Artificial Sequence
  CASP8 sgRNA target sequence
cacagcattagggacaggaa

SEQ ID NO: 9 - DNA, 20 bp, Artificial Sequence
  NLRP1 sgRNA target sequence
ggagttaagagggtgtctgg

SEQ ID NO: 10 - DNA, 20 bp, Artificial Sequence
  MAP3K20(ZAKa) sg1 target sequence
tgtatggttatggaaccgag

SEQ ID NO: 11 - DNA, 21 bp, Artificial Sequence
  MAP3K20(ZAKa) sg4 target sequence
tgcatggaccggaagacgatg

SEQ ID NO: 12 - DNA, 20 bp, Artificial Sequence
  ZNF598 target sequence
cggcactcgcgccggaacga

SEQ ID NO: 13 - DNA, 20 bp, Artificial Sequence
  MAPK14(p38a) sg1 target sequence
tgatgaaatgacaggctacg

SEQ ID NO: 14 - DNA, 20 bp, Artificial Sequence
  MAPK11(p38b) sg1 target sequence
cgacgagcacgttcaattcc

SEQ ID NO: 15 - DNA, 20 bp, Artificial Sequence
  MAPK11(p38b) sg2 target sequence
actcggccgggatcatccac

SEQ ID NO: 16 - protein, 7 aa, Artificial Sequence
  Target motif
PTSTAVL

SEQ ID NO: 17 - protein, 800 aa, Homo sapiens
MSSLGASFVQIKFDDLQFFENCGGGSFGSVYRAKWISQDKEVAVKKLLKIEKEAEILSVLSHRNIIQFYGVILEPPNYGIVTEYASLGSLYDYINSNRSEEMDMDHIMTWATDVAKGMHYLHMEAPVKVIHRDLKSRNVVIAADGVLKICDFGASRFHNHTTHMSLVGTFPWMAPEVIQSLPVSETCDTYSYGVVLWEMLTREVPFKGLEGLQVAWLVVEKNERLTIPSSCPRSFAELLHQCWEADAKKRPSFKQIISILESMSNDTSLPDKCNSFLHNKAEWRCEIEATLERLKKLERDLSFKEQELKERERRLKMWEQKLTEQSNTPLLPSFEIGAWTEDDVYCWVQQLVRKGDSSAEMSVYASLFKENNITGKRLLLLEEEDLKDMGIVSKGHIIHFKSAIEKLTHDYINLFHFPPLIKDSGGEPEENEEKIVNLELVFGFHLKPGTGPQDCKWKMYMEMDGDEIAITYIKDVTFNTNLPDAEILKMTKPPFVMEKWIVGIAKSQTVECTVTYESDVRTPKSTKHVHSIQWSRTKPQDEVKAVQLAIQTLFTNSDGNPGSRSDSSADCQWLDTLRMRQIASNTSLQRSQSNPILGSPFFSHFDGQDSYAAAVRRPQVPIKYQQITPVNQSRSSSPTQYGLTKNFSSLHLNSRDSGFSSGNTDTSSERGRYSDRSRNKYGRGSISLNSSPRGRYSGKSQHSTPSRGRYPGKFYRVSQSALNPHQSPDFKRSPRDLHQPNTIPGMPLHPETDSRASEEDSKVSEGGWTKVEYRKKPHRPSPAKTNKERARGDHRGWRNF

SEQ ID NO: 18 - protein, 1473 aa, Homo sapiens
MAGGAWGRLACYLEFLKKEELKEFQLLLANKAHSRSSSGETPAQPEKTSGMEVASYLVAQYGEQRAWDLALHTWEQMGLRSLCAQAQEGAGHSPSFPYSPSEPHLGSPSQPTSTAVLMPWIHELPAGCTQGSERRVLRQLPDTSGRRWREISASLLYQALPSSPDHESPSQESPNAPTSTAVLGSWGSPPQPSLAPREQEAPGTQWPLDETSGIYYTEIREREREKSEKGRPPWAAVVGTPPQAHTSLQPHHHPWEPSVRESLCSTWPWKNEDFNQKFTQLLLLQRPHPRSQDPLVKRSWPDYVEENRGHLIEIRDLFGPGLDTQEPRIVILQGAAGIGKSTLARQVKEAWGRGQLYGDRFQHVFYFSCRELAQSKVVSLAELIGKDGTATPAPIRQILSRPERLLFILDGVDEPGWVLQEPSSELCLHWSQPQPADALLGSLLGKTILPEASFLITARTTALQNLIPSLEQARWVEVLGFSESSRKEYFYRYFTDERQAIRAFRLVKSNKELWALCLVPWVSWLACTCLMQQMKRKEKLTLTSKTTTTLCLHYLAQALQAQPLGPQLRDLCSLAAEGIWQKKTLFSPDDLRKHGLDGAIISTFLKMGILQEHPIPLSYSFIHLCFQEFFAAMSYVLEDEKGRGKHSNCIIDLEKTLEAYGIHGLFGASTTRFLLGLLSDEGEREMENIFHCRLSQGRNLMQWVPSLQLLLQPHSLESLHCLYETRNKTFLTQVMAHFEEMGMCVETDMELLVCTFCIKFSRHVKKLQLIEGRQHRSTWSPTMVVLFRWVPVTDAYWQILFSVLKVTRNLKELDLSGNSLSHSAVKSLCKTLRRPRCLLETLRLAGCGLTAEDCKDLAFGLRANQTLTELDLSFNVLTDAGAKHLCQRLRQPSCKLQRLQLVSCGLTSDCCQDLASVLSASPSLKELDLQQNNLDDVGVRLLCEGLRHPACKLIRLGLDQTTLSDEMRQELRALEQEKPQLLIFSRRKPSVMTPTEGLDTGEMSNSTSSLKRQRLGSERAASHVAQANLKLLDVSKIFPIAEIAEESSPEVVPVELLCVPSPASQGDLHTKPLGTDDDFWGPTGPVATEVVDKEKNLYRVHFPVAGSYRWPNTGLCFVMREAVTVEIEFCVWDQFLGEINPQHSWMVAGPLLDIKAEPGAVEAVHLPHFVALQGGHVDTSLFQMAHFKEEGMLLEKPARVELHHIVLENPSFSPLGVLLKMIHNALRFIPVTSVVLLYHRVHPEEVTFHLYLIPSDCSIRKAIDDLEMKFQFVRIHKPPPLTPLYMGCRYTVSGSGSGMLEILPKELELCYRSPGEDQLFSEFYVGHLGSGIRLQVKDKKDETLVWEALVKPGDLMPATTLIPPARIAVPSPLDAPQLLHFVDQYREQLIARVTSVEVVLDKLHGQVLSQEQYERVLAENTRPSQMRKLFSLSQSWDRKCKDGLYQALKETHPHLIMELWEKGSKKGLLPLSS

SEQ ID NO: 19 - DNA, 4422 bp, Homo sapiens
atggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggctggaccagacaactctgagtgatgagatgaggcaggaactgagggccctggagcaggagaaacctcagctgctcatcttcagcagacggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggccatagatgatctagaaatgaaattccagtttgtgcgaatccacaagccacccccgctgaccccactttatatgggctgtcgttacactgtgtctgggtctggttcagggatgctggaaatactccccaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctga

SEQ ID NO: 20 - DNA, 2403 bp, Homo sapiens
atgtcgtctctcggtgcctcctttgtgcaaattaaatttgatgacttgcagttttttgaaaactgcggtggaggaagttttgggagtgtttatcgagccaaatggatatcacaggacaaggaggtggctgtaaagaagctcctcaaaatagagaaagaggcagaaatactcagtgtcctcagtcacagaaacatcatccagttttatggagtaattcttgaacctcccaactatggcattgtcacagaatatgcttctctgggatcactctatgattacattaacagtaacagaagtgaggagatggatatggatcacattatgacctgggccactgatgtagccaaaggaatgcattatttacatatggaggctcctgtcaaggtgattcacagagacctcaagtcaagaaacgttgttatagctgctgatggagtattgaagatctgtgactttggtgcctctcggttccataaccatacaacacacatgtccttggttggaactttcccatggatggctccagaagttatccagagtctccctgtgtcagaaacttgtgacacatattcctatggtgtggttctctgggagatgctaacaagggaggtcccctttaaaggtttggaaggattacaagtagcttggcttgtagtggaaaaaaacgagagattaaccattccaagcagttgccccagaagttttgctgaactgttacatcagtgttgggaagctgatgccaagaaacggccatcattcaagcaaatcatttcaatcctggagtccatgtcaaatgacacgagccttcctgacaagtgtaactcattcctacacaacaaggcggagtggaggtgcgaaattgaggcaactcttgagaggctaaagaaactagagcgtgatctcagctttaaggagcaggagcttaaagaacgagaaagacgtttaaagatgtgggagcaaaagctgacagagcagtccaacaccccgctgctgccttcctttgagattggtgcatggacggaagacgatgtgtattgttgggttcagcagctcgtcagaaaaggtgactcttcagcagagatgagtgtatatgcaagcttgtttaaagaaaacaacattacagggaagcggctgctgctgctggaggaagaagacctgaaagacatgggcattgtctccaaggggcatatcattcacttcaagtcagccattgagaaattaacccatgattacataaatttgtttcacttcccaccactaattaaggactcaggaggtgaacctgaagaaaatgaggaaaaaatagtgaacctggaactggtttttggttttcacttgaaaccaggaactggcccacaggattgtaagtggaaaatgtatatggagatggatggggatgaaattgcaataacctacataaaagatgtgacattcaacactaacctacctgatgcggagattttaaagatgacaaagccaccatttgtaatggagaagtggattgtaggaatagcaaaaagtcagactgtggagtgcactgtcacatatgagagtgatgttagaactccaaaaagcactaaacatgtccattcgattcagtggagtagaacaaaacctcaggatgaagtgaaagcagtccaacttgccattcagacattattcaccaattcagatggcaaccctggaagcaggtccgactcaagtgctgattgccagtggttagatactctgaggatgcggcagattgcatccaacacttctttacagcgttcccagagcaatcctattctggggtcaccgttcttctcacactttgatggccaggattcctacgctgctgctgtgagacggccccaggtgcccattaagtatcaacagattacacctgtgaaccagtccagaagctcgtctcctactcagtatggactgaccaaaaacttctcttccctacatctcaactctagggacagtggcttttccagtggcaatactgacacctcttcagagaggggtcgatactcagacagaagcaggaacaaatatggacgtggtagtatatcactcaattcttctcctagaggaagatacagtggaaagagtcagcattccactccttcaagaggaagataccctggaaagttctacagggtttctcagtcagcactcaatcctcaccagtcgcctgacttcaagagaagccccagggacctccaccaacccaacaccataccagggatgcctttgcaccctgagactgactcaagagccagtgaagaggacagcaaagtcagcgaagggggctggacaaaagtggaataccggaaaaagccccacaggccatctcccgccaaaaccaataaagagagagccagaggggaccaccgtggatggagaaacttttga